(19) United States
(12) Patent Application Publication
(10) Pub. No.: US 2025/0345559 A1
(43) Pub. Date: Nov. 13, 2025

(54) SYSTEMS AND METHODS FOR HYDRATING A FLEXIBLE ELONGATED DEVICE

(71) Applicant: Intuitive Surgical Operations, Inc., Sunnyvale, CA (US)

(72) Inventors: Majeed A. Foad, Sunnyvale, CA (US); Alexander A. Arazawa, Santa Clara, CA (US); Joseph D. Bogusky, San Jose, CA (US); Joseph R. Callol, San Francisco, CA (US); Francis T. Macnamara, San Jose, CA (US)

(21) Appl. No.: 19/201,343

(22) Filed: May 7, 2025

Related U.S. Application Data
(60) Provisional application No. 63/644,308, filed on May 8, 2024.
(60) Provisional application No. 63/644,404, filed on May 8, 2024.

(51) Int. Cl. A61M 25/00

(52) U.S. Cl. CPC A61M 25/0026; A61M 25/0045; A61M 2025/0037; A61M 2025/0046; A61M 2205/0216; A61M 2205/0222; A61M 2205/0238; A61M 2205/0283; A61M 2205/3331; A61M 2205/50

(57) ABSTRACT

A system may comprise a flexible elongated device including a fluid channel and a pore extending between the fluid channel and a surface of the flexible elongated device and a lubricious layer extending over at least a portion of the surface of the flexible elongated device. The system may also comprise a fluid system coupled to the flexible elongated device and a control system configured to cause fluid to be released by the fluid system to the fluid channel of the flexible elongated device. The fluid in the fluid channel flows through the pore to transition the lubricious layer from a dehydrated condition to a hydrated condition.

### RELATED APPLICATIONS

This patent application claims priority to and benefit of U.S. Provisional Patent Application 63/644,308, entitled “SYSTEMS AND METHODS FOR HYDRATING A FLEXIBLE ELONGATED DEVICE,” filed May 8, 2024 and U.S. Provisional Patent Application 63/644,404, entitled “FLEXIBLE ELONGATED DEVICE WITH A LUBRICIOUS LAYER AND METHODS OF USE”, filed May 8, 2024 which are incorporated by reference herein in their entirety. This patent application is related to U.S. Provisional Patent Application 63/644,351, entitled “SYSTEMS AND METHODS FOR DETECTING A HYDRATION CONDITION OF A FLEXIBLE ELONGATED DEVICE,” filed May 8, 2024 which is incorporated by reference herein in its entirety.

### FIELD

The present disclosure is related to a flexible elongated device with a lubricious layer and to systems and methods for hydrating a flexible elongated device.

### BACKGROUND

Minimally invasive medical techniques are intended to reduce the amount of tissue that is damaged during medical procedures, thereby reducing patient recovery time, discomfort, and harmful side effects. Such minimally invasive techniques may be performed through natural orifices in a patient anatomy or through one or more surgical incisions. Through these natural orifices or incisions, an operator may insert a minimally invasive medical instrument (including surgical, diagnostic, therapeutic, and/or biopsy instruments) to reach a target tissue location. One such minimally invasive technique is to use a flexible elongated device, such as a flexible catheter, bronchoscope, or endoscope, that can be inserted into anatomic passageways and navigated toward a region of interest within the patient anatomy. When navigating passageways within a patient anatomy, the flexible elongated device may experience friction with the passageway walls resulting in difficulty navigating the device and reaching a target anatomical location. Systems and methods to reduce friction between the flexible elongated device and the patient anatomy are needed.

### SUMMARY

The following presents a simplified summary of various examples described herein and is not intended to identify key or critical elements or to delineate the scope of the claims.

In some examples, a system may comprise a flexible elongated device including a fluid channel and a pore extending between the fluid channel and a surface of the flexible elongated device and a lubricious layer extending over at least a portion of the surface of the flexible elongated device. The system may also comprise a fluid system coupled to the flexible elongated device and a control system configured to cause fluid to be released by the fluid system to the fluid channel of the flexible elongated device. The fluid in the fluid channel flows through the pore to transition the lubricious layer from a dehydrated condition to a hydrated condition.

In some examples, a system may comprise a flexible elongated device configured to couple to a robotically-assisted manipulator assembly. The flexible elongated device includes a lubricious layer extending over at least a portion of a surface of the flexible elongated device. The system may also comprise a hydration coupling system connected to the robotically-assisted manipulator assembly. The system may also comprise a control system configured to cause a fluid to be released through the hydration coupling system into contact with an external surface of the flexible elongated device to transition the lubricious layer from a dehydrated condition to a hydrated condition.

In some examples, a method may comprise causing a fluid to be released by a fluid system to a fluid channel of a flexible elongated device coupled to the fluid system and causing the fluid to flow through a pore of the flexible elongated device extending between the fluid channel and a surface of the flexible elongated device to transition a lubricious layer extending over at least a portion of the flexible elongated device from a dehydrated condition to a hydrated condition.

In some examples, a method comprises causing a fluid to be released through a hydration coupling system into contact with a lubricious layer extending over at least a portion of a flexible elongated device and transitioning the lubricious layer from a dehydrated condition to a hydrated condition.

In some examples, a flexible elongated device may comprise a flexible elongated body defining a lumen. The flexible elongated body includes an outer surface with a surface texture. The flexible elongated device may also comprise a lubricious layer extending over at least a portion of the outer surface and configured to transition from an anhydrous condition to a hydrated condition when exposed to a liquid. The lubricious layer in the hydrated condition may be thicker than the lubricious layer in the anhydrous condition resulting from expansion of the lubricious layer. The lubricious layer in the hydrated condition may form a coating texture on an outer surface of the lubricious layer based on the surface texture of the flexible elongated body under the lubricious layer.

It is to be understood that both the foregoing general description and the following detailed description are illustrative and explanatory in nature and are intended to provide an understanding of the present disclosure without limiting the scope of the present disclosure. In that regard, additional aspects, features, and advantages of the present disclosure will be apparent to one skilled in the art from the following detailed description.

### BRIEF DESCRIPTIONS OF THE DRAWINGS

FIG. 1A illustrates a flexible elongated device including a fluid system, according to some examples.

FIG. 1B illustrates a cross-sectional view of the flexible elongated device of FIG. 1A, according to some examples.

FIG. 1C illustrates a cross-sectional view of a flexible elongated device, according to some examples.

FIG. 2A illustrates a flexible elongated device including a lubricious layer and a fluid system, according to some examples.

FIG. 2B illustrates a cross-sectional view of the flexible elongated device of FIG. 2A, according to some examples.

FIG. 2C illustrates a cross-sectional view of a flexible elongated device, according to some examples.

FIG. 2D illustrates a cross-sectional view of a flexible elongated device, according to some examples.

FIG. 3A illustrates a side view of a flexible elongated device including a plurality of pores, according to some examples.

FIG. 3B illustrates a side view of a flexible elongated device including a plurality of pores, according to some examples.

FIG. 3C illustrates a side view of a flexible elongated device including a plurality of pores, according to some examples.

FIG. 3D illustrates a side view of a flexible elongated device including a plurality of pores, according to some examples.

FIG. 3E illustrates a side view of a flexible elongated device including a directional pore, according to some examples.

FIG. 4 illustrates a cross-sectional view of a flexible elongated device including an active layer, according to some examples.

FIG. 5 illustrates a manipulator assembly with a flexible elongated device extending into a patient anatomy, according to some examples.

FIG. 6 illustrates a manipulator assembly with a flexible elongated device extending into a patient anatomy, according to some examples.

FIG. 7 illustrates a manipulator assembly with a flexible elongated device extending into a patient anatomy, according to some examples.

FIG. 8 illustrates a manipulator assembly with a flexible elongated device extending into a patient anatomy, according to some examples.

FIG. 9 is a flowchart illustrating a method of hydrating a flexible elongated device, according to some examples.

FIG. 10 illustrates a packaging hydration system, according to some examples.

FIG. 11 illustrates a flexible elongated device including a lubricious layer, according to some examples.

FIG. 12 illustrates a flexible elongated device including a lubricious layer, according to some examples.

FIG. 13A illustrates a flexible elongated device including a surface texture, according to some examples.

FIG. 13B illustrates a recessed microstructure, according to some examples,

FIG. 13C illustrates a raised microstructure, according to some examples.

FIG. 13D illustrates the flexible elongated device of FIG. 13A with a lubricious layer, according to some examples.

FIG. 14A illustrates a flexible elongated device including a surface texture with patterned microstructures, according to some examples.

FIG. 14B illustrates the flexible elongated device of FIG. 14A with a lubricious layer, according to some examples.

FIG. 15A illustrates a flexible elongated device including a surface texture with a ribbed pattern, according to some examples.

FIG. 15B illustrates the flexible elongated device of FIG. 15A with a lubricious layer, according to some examples.

FIG. 16A illustrates a flexible elongated device including a surface texture with a spiral pattern, according to some examples.

FIG. 16B illustrates the flexible elongated device of FIG. 16A with a lubricious layer, according to some examples.

FIG. 17A illustrates a flexible elongated device including a surface texture with a radial pattern, according to some examples.

FIG. 17B illustrates the flexible elongated device of FIG. 17A with a lubricious layer, according to some examples.

FIG. 18A illustrates a flexible elongated device including a surface texture with a woven pattern, according to some examples.

FIG. 18B illustrates the flexible elongated device of FIG. 18A with a lubricious layer, according to some examples.

FIG. 19 illustrates a flexible elongated device including a lubricious layer with contoured features formed in the lubricious layer, according to some examples.

FIG. 20 is a flowchart illustrating a method of using a flexible elongated device, according to some examples.

FIG. 21 illustrates an example of a flexible elongated device in a patient anatomy near a target tissue, according to some examples.

FIG. 22 is a simplified diagram of a medical system, according to some examples.

FIG. 23A is a simplified diagram of a medical instrument system, according to some examples.

FIG. 23B is a simplified diagram of a medical instrument including a medical tool within a flexible elongated device, according to some examples.

Embodiments of the present disclosure and their advantages are best understood by referring to the detailed description that follows. It should be appreciated that like reference numerals are used to identify like elements illustrated in one or more of the figures, wherein showings therein are for purposes of illustrating embodiments of the present disclosure and not for purposes of limiting the same.

### DETAILED DESCRIPTION

The systems described herein may include flexible elongated devices (e.g., catheters, bronchoscopes, or endoscopes). As flexible elongated devices navigate anatomic passageways, they may experience friction with the inner wall of the passageways or with components of manipulator systems to which they may be attached. Friction may compromise control and navigation of the flexible elongated device through increased resistance, stick-slip behavior, prolapse of the device externally or internally of the patient, or an inability to reach a target location. Flexible elongated devices may be covered with a lubricious layer or coating, such as hydrophilic material, to reduce or eliminate the issues associated with friction. In some types of anatomic passageways, such as cardiovascular or neurovascular passageways, naturally-present body fluids or mucous may activate a lubricious layer to augment lubrication provided by the anatomy. In contrast, flexible elongated devices navigating through other types of anatomic passageways (e.g., lung passageways) may be exposed to flowing air that dries the flexible elongated devices and increases the coefficient of friction of the flexible elongated device. Lubricious layers may be activated by non-anatomic sources of hydration to increase lubrication in these drier anatomic environments. The systems described herein may be used to deliver a hydration fluid to a flexible elongated device, which may include a lubricious layer.

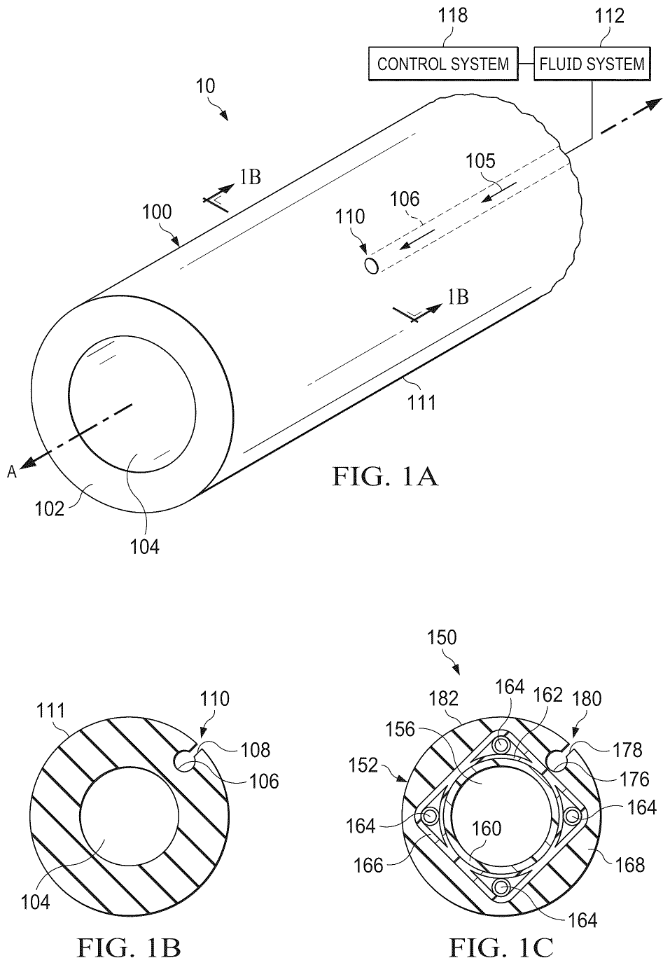

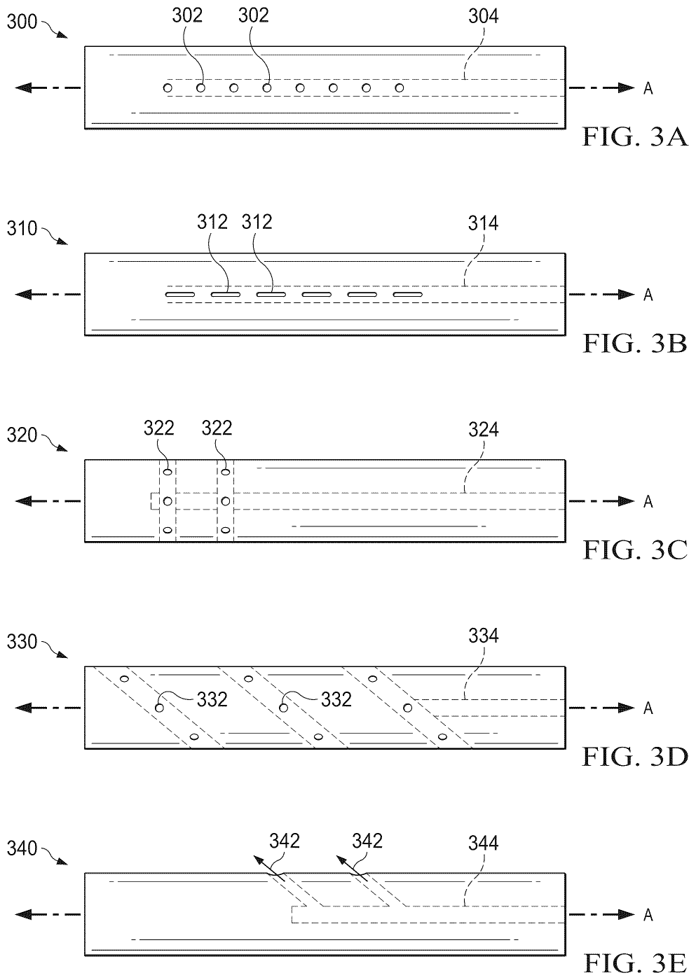

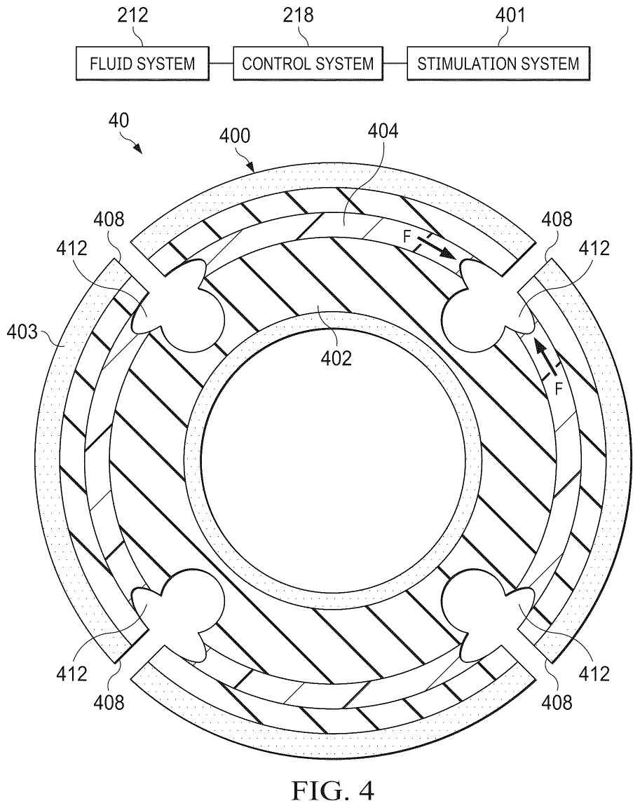

FIG. 1A illustrates a system 10 including a flexible elongated device 100, a fluid system 112, and a control system 118. The flexible elongated device 100 may include a flexible elongated body 102 extending along a longitudinal axis A. The flexible elongated body 102 defines a lumen 104 through which, for example, tools may be inserted or fluids may be introduced or evacuated. A fluid channel 106 extends through the flexible elongated body 102 to convey a hydration fluid 105 such as water, saline, or another hydrating liquid or vapor. As shown in the cross-sectional view of FIG. 1B, the fluid channel 106 may extend through the flexible elongated body 102. A pore 108 may provide a conduit between the fluid channel 106 and an opening 110 in an outer surface 111 of the flexible elongated body 102. In some examples, the pore may be generally round and may have a diameter between approximately 5 and 100 μm. In this example, the pore 108 may extend generally orthogonal to the longitudinal axis A, but in other examples (see, FIG. 3E) the pores may be angled in non-orthogonal directions to direct the hydrating fluid toward a proximal or distal end of the flexible elongate device. In alternative examples, the fluid channel may extend through the lumen 104 or may be attached to and extend along an outer surface of the flexible elongated body 102.

A fluid system 112 may be coupled to the fluid channel 106. In some examples, the fluid system 112 may include a reservoir containing the hydrating fluid 105. In alternative examples, the fluid system 112 may couple to and receive the hydrating fluid 105 from centralized system such as a building water system. In some examples, the fluid system may include pumps, valves, syringes, conduit, fluid reservoirs, couplings, or other flow control mechanisms to provide control of the activation and speed of flow of the hydrating fluid 105 from the reservoir to the fluid channel 106. Actuation, speed of flow, and other operations of the fluid system 112 may be controlled by a control system 118. The control system 118 may receive an indication that that the flexible elongated device 100 is in an unlubricated or under-lubricated condition, and responsive to receipt of the indication may actuate the fluid system 112 to release hydrating fluid 105 from the fluid system 112 into the fluid channel 106. The indication may be received, for example, from a user input at a user input device of a master assembly (e.g. master assembly 806) or from a hydration detection system (e.g. hydration detection system 220). The hydrating fluid 116 may flow through the fluid channel 106, the pore 108, and the opening 110 to the surface 111 to provide lubrication to the flexible elongated device 100.

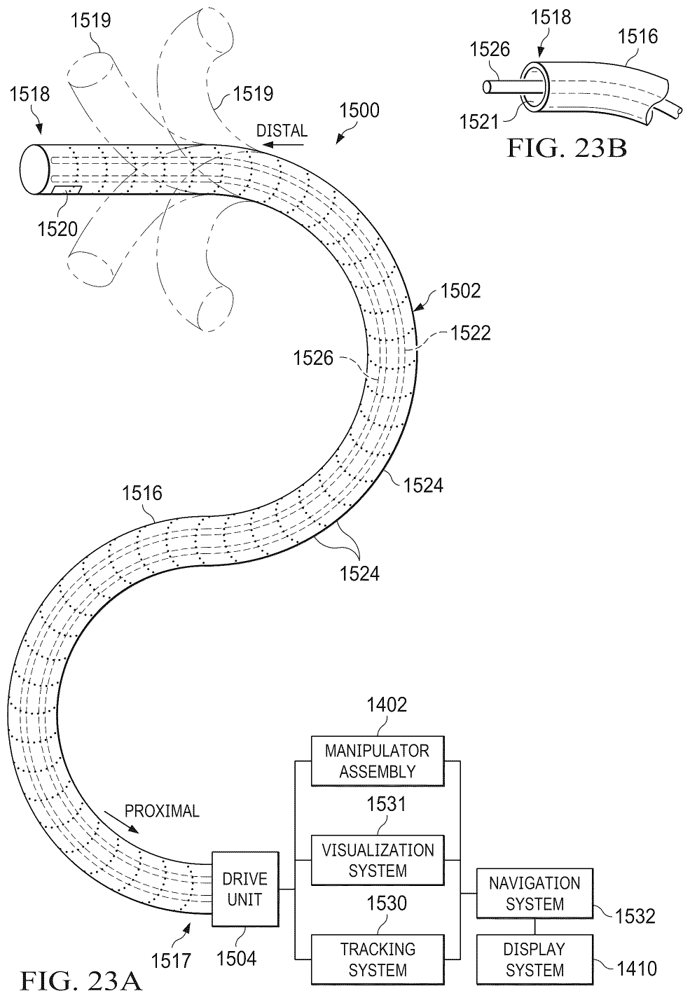

In some examples, the flexible elongated device 100 may be a component of a robotically assisted medical instrument system or a manually-controlled medical instrument system that controls articulation and insertion/retraction of the flexible elongated device 100. An example of a medical instrument system including a flexible elongated device that is bendable and steerable in multiple degrees of freedom is described below in FIGS. 23A and 23B (e.g., medical instrument system 1500). In some examples, the control system 118 may be a control system (e.g. control system 1412) of a robotically-assisted medical system.

FIG. 1C is a cross-sectional view of a flexible elongated device 150 that may be substantially similar to the flexible elongated device 100, with differences as described. The flexible elongated device 150 includes a flexible elongated body 152 with a flexible liner layer 160 defining a lumen 156 through which, for example, tools may be inserted or fluids may be introduced or evacuated. In some examples, the liner layer 160 may be formed from a polymer material such as polytetrafluoroethylene (PTFE) or another fluoropolymer. The flexible elongated body 152 may also include a support layer 162 extending along at least a portion of the liner layer 160. The support layer 162 may include a woven material including for example, coiled polymer fibers, braided polymer fibers, coiled metal fibers, braided metal fibers or may include a non-woven material such as a laser cut hypo tube. The flexible elongated body 152 may also include elongated control members 164 extending along the support layer 162 for controlling articulation and steering of the flexible elongated device 150. Although four control members are shown, in various example, the number of control members may be greater than or less than four. The flexible elongated body 102 may also include a support layer 166 extending around the control members 164. The support layer 166 may include a woven material including for example, coiled polymer fibers, braided polymer fibers, coiled metal fibers, braided metal fibers or may include a non- woven material such as a laser cut hypo tube. The flexible elongated body 152 may also include a flexible elastomeric layer 168. In some examples, the flexible elastomeric layer may include an elongated thermoplastic elastomer that extends around the support layer 166. The flexible elastomeric layer may, for example, be laminated to the support layer 166 by heat shrink. For example, a heat shrink tubing, such as a fluorinated ethylene propylene tubing, may be applied around the flexible elastomeric layer 168 and then heated. The heat shrink tubing may be removed after the flexible elastomeric layer 168 is laminated onto and into the support layer 166. A fluid channel 176 may be formed in and extend longitudinally through the flexible elastomeric layer 168. A pore 178 may extend from the fluid channel 176, through the flexible elastomeric layer 168 to an opening 180 on the outer surface 182 of the flexible elastomeric layer 168. In some examples, the fluid channel and pore may be arranged within the flexible elastomeric layer to avoid interference with the support layers and control members.

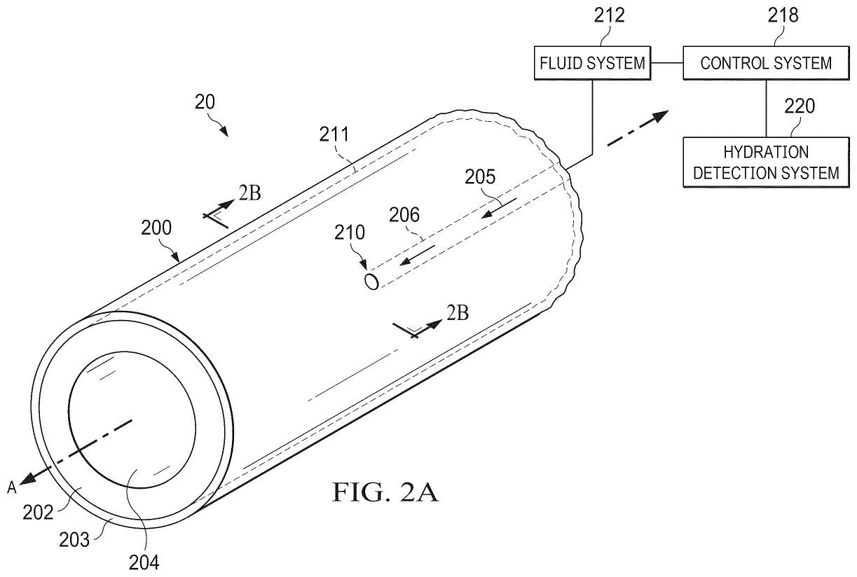

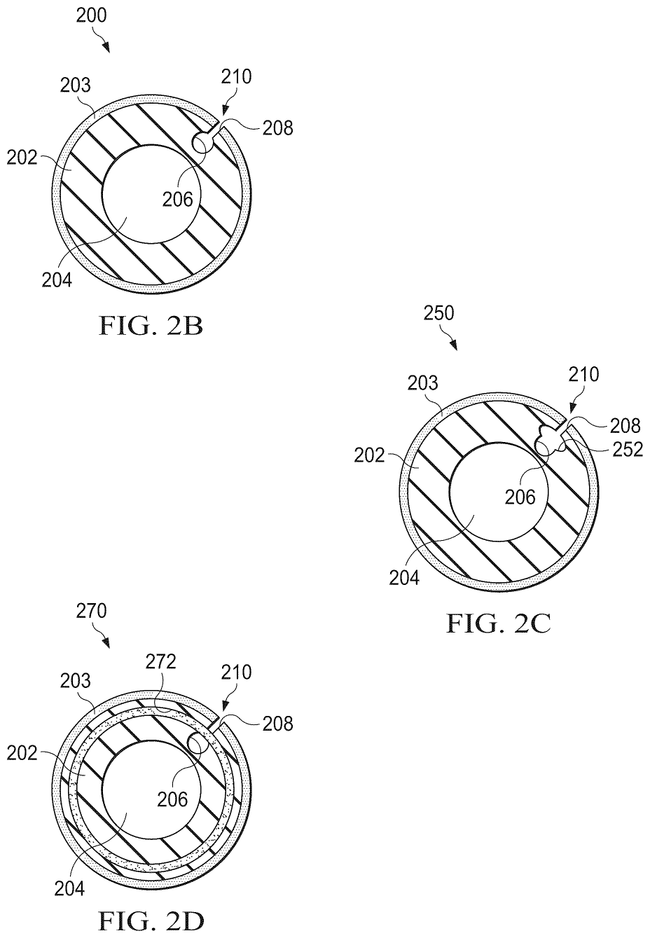

FIG. 2A illustrates a system 20 including a flexible elongated device 200, a fluid system 212, and a control system 218. The flexible elongated device 200, the fluid system 212, and the control system 218 may be substantially similar to the flexible elongated device 100, the fluid system 112, and the control system 118, respectively, with differences as described. The flexible elongated device 200 may include a flexible elongated body 202 extending along a longitudinal axis A. The flexible elongated body 202 defines a lumen 204 through which, for example, tools may be inserted or fluids may be introduced or evacuated. A fluid channel 206 extends through the flexible elongated body 202 to convey a hydration fluid 205 such as water, saline, or another hydrating liquid or vapor. In other examples the fluid channel 206 may extend through the lumen 204 or along an outer surface 211 of the flexible elongated body 202. In this example, a lubricious layer 203 extends along at least a portion of the length of the outer surface 211 of the flexible elongated body 202. The lubricious layer 203 may include, for example, a hydrophilic substance or material that may promote lubricity thereby reducing or preventing stick-slip or irregular sliding behaviors as the flexible elongated device 200 is introduced into a patient anatomy. In some examples, the lubricious layer 203 may have a hydrated or activated condition in which the hydrophilic material is hydrated and/or retentive of fluid. The lubricious layer 203 may also have a dehydrated condition in which the hydrophilic material is in an inactivated, anhydrous, or dehydrated. The lubricious layer in the hydrated condition may be thicker than the lubricious layer in the dehydrated condition, resulting from expansion of the lubricious layer. The lubricious layer 203 may be applied to the outer surface 211 of the flexible elongated body 202 by dip coating, spray-on application, wrap material application, wipe-on application, or as a tubular overlay, for example.

In some examples, a dehydrated threshold level may be a hydration level at or below which a dehydrated condition is indicated. A hydrated threshold level may be a hydration level at or above which a hydrated condition is indicated. For example a hydrated threshold level may be associated with sufficient lubricity for performing a procedure with the flexible elongated device. In some examples, the dehydrated threshold level and the hydrated threshold level may be the same such that the dehydrated condition exists at or below the threshold level and the hydrated condition exists above the threshold level. In other examples, the dehydrated threshold level may be below the hydrated threshold level. In such an example, the hydrated threshold level may be associated with a fully hydrated lubricious layer and the dehydrated threshold level may be associated with a lubricious layer insufficiently hydrated to perform a procedure with the flexible elongated device. Thus, between the hydrated threshold level and the dehydrated threshold level, the lubricious layer may be sufficiently hydrated to perform a procedure but may not be optimally or maximally hydrated. A dehydrated condition of the lubricious layer may trigger an action such as warning the operator or re-hydrating the lubricious layer. Various examples of hydration conditions are disclosed below. In some examples, the lubricious layer 203 may include a visible pigment or dye that imparts an identifying color to the lubricious layer. The visible pigment may identify which portion of the flexible elongated device include the lubricious layer and thus will transition to a hydrated condition when hydrated. The visible pigment may also or alternatively indicate wear or delamination of the lubricious layer, signaling for example that the lubricious layer should be reapplied to the device discarded. In some alternative examples, the lubricious layer may include a hydrophobic material.

The system 20 may optionally include or operate in cooperation with a hydration detection system 220. The hydration detection system 220 may detect a hydration indicator for the lubricious layer 203 and evaluate the hydration indicator to determine the hydration condition (e.g., level of hydration) of the flexible elongated device 200 or the lubricious layer 203. Various examples of hydration detection systems are disclosed in U.S. Provisional Patent Application 63/644,351, “SYSTEMS AND METHODS FOR DETECTING A HYDRATION CONDITION OF A FLEXIBLE ELONGATED DEVICE,” filed May 8, 2024, which is incorporated by reference herein, in its entirety, for all purposes. The hydration detection system 220 may communicate the hydration condition of the flexible elongated device 200 or the lubricious layer 203 to the control system 218 which may, in turn, control the fluid system 212 to dispense hydrating fluid 205 through the fluid channel 206.

As shown in the cross-sectional view of FIG. 2B, the fluid channel 206 may extend through the flexible body 202. A pore 208 may provide a conduit between the fluid channel 206, an opening 110 in an outer surface 111 of the flexible elongated body 102, and to and/or through the lubricious layer 203. In some examples, the pore 208 may be generally round and may have a diameter between approximately 5 and 100 μm. The fluid system 212 may be coupled to the fluid channel 206 and may work in coordination with the control system 218, as previously described. For example, the control system 218 may receive an indication that that the lubricious layer 203 is in a dehydrated condition, and responsive to receipt of the indication may actuate the fluid system 212 to release hydrating fluid 205 from the fluid system 212 into the fluid channel 206. The indication may be received, for example, from a user input at a user input device of a master assembly (e.g. master assembly 806) or from a hydration detection system (e.g. hydration detection system 220). The hydrating fluid 205 may flow through the fluid channel 206, the pore 208, and to the lubricious layer 203 to provide lubrication to the flexible elongated device 200. The hydrating fluid 205 may be dispensed to the lubricious layer 203 to transition the lubricious layer 203 from a dehydrated condition to a hydrated condition or to maintain the lubricious layer 203 above the dehydrated threshold level.

The size of the pore may be selected to achieve a desired result. For example, the pore and opening may have a diameter larger than the thickness of the lubricious layer so that in a hydrated condition, the pore and opening remain unobstructed and fluid may pass through the opening. In other examples, the pore and opening may have a diameter smaller than the thickness of the lubricious layer so that in a hydrated condition, the pore and opening are obstructed and fluid may not pass through the opening until the lubricious layer becomes dehydrated.

In some examples, the flexible elongated device 200 may be a component of a robotically assisted medical instrument system or a manually-controlled medical instrument system that controls articulation and insertion/retraction of the flexible elongated device 200. An example of a medical instrument system including a flexible elongated device that is bendable and steerable in multiple degrees of freedom is described below in FIGS. 23A and 23B (e.g., system 1500). In some examples, the control system 218 may be a control system (e.g. control system 1412) of a robotically assisted medical system.

FIG. 2C is a cross-sectional view of a flexible elongated device 250 that may be substantially similar to the flexible elongated device 200, with differences as described. In this example, a hydration reservoir 252 may be a pocket extending between the fluid channel 206 and the pore 208 to retain hydration fluid 205 near the lubricious layer 203. The fluid 205 may be absorbed by the lubricious layer 203 from the hydration reservoir 252, over time, to main the hydration condition of the lubricious layer. In some examples, the hydration reservoir may be a cavity in a flexible elastomeric layer of the body 202. The pores and/or hydration reservoir may be formed by a laser drilling or cutting into the body 202 and may be formed before or after deposition of the lubricious layer 203. In some examples, a hydration reservoir may be used in embodiments such as FIG. 1 that do not include a lubricious layer.

FIG. 2D is a cross-sectional view of a flexible elongated device 270 that may be substantially similar to the flexible elongated device 200, with differences as described. In this example, a hydration reservoir 272 may include an open cell foam member extending between the fluid channel 206 and the pore 208 to retain hydration fluid 205 near the lubricious layer 203. The fluid 205 may be absorbed by the lubricious layer 203 from the hydration reservoir 272, over time, to main the hydration condition of the lubricious layer. In some examples, an open cell foam member may include a polymer sponge. In some examples, the hydration reservoir may be embedded within the flexible elastomeric layer of the body 202 and may be in fluid communication with the lubricious layer 203 via the pore 208. In other examples, the hydration reservoir may extend between the body 202 and the lubricious layer 203 with the open cell foam member in direct contact with the lubricious layer 203.

In various examples, a plurality of pores may be used to provide hydration to a flexible elongated device. The pores may have a variety of regular or irregular shapes and may be arranged in a variety of regular or irregular patterns. In various examples, pores may be arranged along a single side of the flexible elongated device to provide directional hydration or hydration directed toward targeted regions of a lubricious layer. In other examples, the pores may extend around the circumference of the flexible elongated device to provide hydration around the circumference of the flexible elongated device. In some examples, pores may be more abundant in a particular region of the flexible elongated device, such as a proximal end region or a distal end region. Examples provided below are for illustration, but other shapes and arrangements of pores may be suitable.

FIG. 3A illustrates a side view of a flexible elongated device 300 (e.g., the flexible elongated device 100, 200) including a plurality of pores 302 (e.g. the pores 108, 208) fed by a fluid channel 304. In this example, the pores 302 may be arranged in a linear series generally parallel to the longitudinal axis A. In this example, the pores 302 may be substantially round or circular. In other examples, the pores may have an oval, elliptical, elongated, or other suitable shape.

FIG. 3B illustrates a side view of a flexible elongated device 310 (e.g., the flexible elongated device 100, 200) including a plurality of pores 312 (e.g. the pores 108, 208) fed by a fluid channel 314. In this example, the pores 312 may be slits or elongated passages arranged in a linear series generally parallel to the longitudinal axis A.

FIG. 3C illustrates a side view of a flexible elongated device 320 (e.g., the flexible elongated device 100, 200) including a plurality of pores 322 (e.g. the pores 108, 208) fed by a fluid channel 324. In this example, the pores 322 may be arranged in a radial series spaced around the longitudinal axis A. In some examples, the pores 322 may form an array of columns and rows.

FIG. 3D illustrates a side view of a flexible elongated device 330 (e.g., the flexible elongated device 100, 200) including a plurality of pores 332 (e.g. the pores 108, 208) fed by a fluid channel 334. In this example, the pores 332 may be arranged in a spiral or helical series around the longitudinal axis A.

FIG. 3E illustrates a side view of a flexible elongated device 340 (e.g., the flexible elongated device 100, 200) including a plurality of pores 342 (e.g. the pores 108, 208) fed by a fluid channel 344. In this example, the pores 342 extend at an angle that is non-orthogonal to the longitudinal axis A. With the pores 342 angled non-orthogonally to the longitudinal axis A, the hydrating fluid 205 may be directed toward a distal end portion of the flexible elongated device 340. In other examples, the pores may be angled to direct the hydrating fluid toward a proximal end portion of the flexible elongated device. In other examples, a flexible elongated device may include a combination of orthogonally and non-orthogonally directed pores.

FIG. 4 illustrates a system 40 including a flexible elongated device 400 (illustrated in a cross-sectional view), the fluid system 212, the control system 218, and a stimulation system 401. The flexible elongated device 400 may be substantially similar to the flexible elongated device 200 with differences as described. The flexible elongated device 400 may include a flexible elongated body 402 and a lubricious layer 403. The flexible elongated body 402 may include an active polymer component 404, which may be a layered component, that changes shape, swells, or otherwise deforms in response to a stimulus from the stimulation system 401. In some examples, the stimulation system 401 may provide a ferroelectric stimulus that induces stress in the active polymer component 404 by changing the polymer polarization with an electric or magnetic field. In some examples, the stimulation system 401 may provide a piezoelectric stimulus that induces a mechanical stress to the active polymer component 404 using an electrical charge. Stimulating the active polymer component 404 may create a compressing force F that squeezes hydrating fluid from the fluid reservoirs 412 and through the pores 408 to dispense the hydrating to the lubricious layer 403.

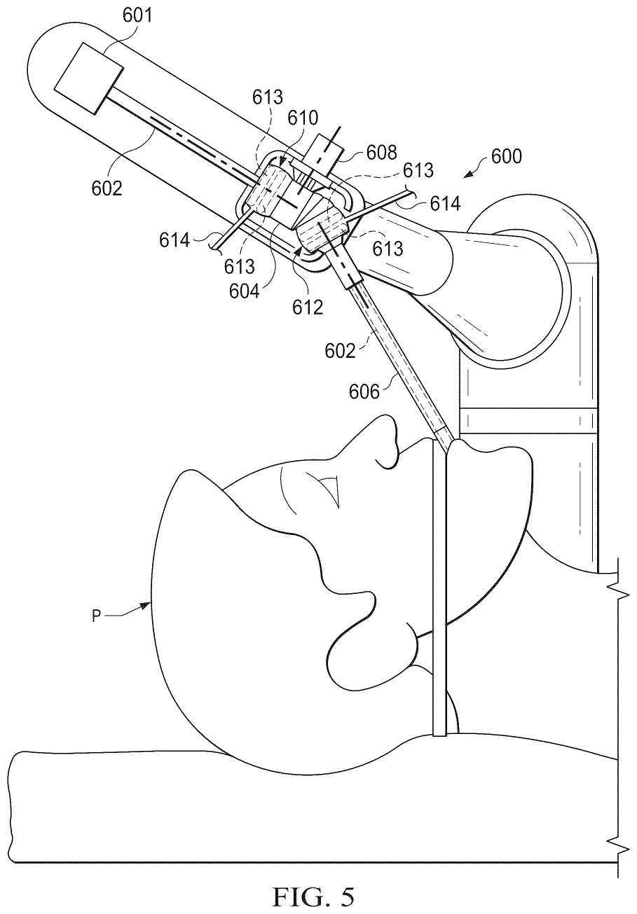

In some examples, a hydrating fluid may be dispensed directly to an outer surface of a flexible elongated device without passing through an interior fluid channel. FIGS. 5-8 illustrate systems and techniques for dispensing the hydrating fluid to the exterior surface of a proximal portion of the flexible elongated device at a location external to the patient anatomy while a distal portion of the flexible elongated device is extended within the patient anatomy. These systems and techniques may reduce or eliminate the need to withdraw the flexible elongated device from the patient anatomy to provide the re-hydration. In some examples, the exterior hydration systems of FIGS. 5-8 may be used in addition to the hydration system of the above described internally delivered hydration systems. In such examples, common or separate fluid systems may be used to provide the hydrating fluid.

FIG. 5 illustrates a manipulator assembly 600 connected to a flexible elongated device 602 (e.g., flexible elongated device 100, 200). In some examples, the flexible elongated device may include a lubricious layer (e.g. a lubricious layer 203), and in other examples a lubricious layer may be omitted. The manipulator assembly 600 may be a robotically-assisted manipulator assembly. For example, the manipulator assembly 600 may be a component (e.g., the manipulator assembly 1402) of a robotically-assisted medical system (e.g., the robotically-assisted medical system 1400). The manipulator assembly 600 may include an instrument carriage 601 to which a proximal end of the flexible elongated device 602 is connected. The manipulator assembly 600 may include a connector device 604 through which the flexible elongated device 602 may extend. In some examples, the connector device 604 may swivel or rotate relative to the manipulator assembly 600. The manipulator assembly may include an anatomic orifice device 606 through which the flexible elongated device 100 may extend to gain entry to the patient anatomy P. The anatomic orifice device 606 may be, for example, an endotracheal tube, a laryngeal mask airway, or a cannula, and may be fixed to patient anatomy P to facilitate insertion of various medical devices. A ventilation port 608 may extend from the connector device 604. The ventilation port 608 may provide a conduit for an external source of air that may be provided to the patient P though the anatomic orifice device 606. The connector device 604 may include a proximal sealing mechanism 610 and a distal sealing mechanism 612. Each sealing mechanism may include a plurality of sealing members 613 to prevent the migration of air or fluid from the connector device 604. The flexible elongated device 602 may extend through the sealing mechanisms 610, 612. A hydration coupling system may connect a fluid system (e.g. the fluid system 112, 212) to a component of the manipulator assembly 600 such as the connector device 604, the ventilation port 608, or the anatomic orifice device 606. In this example, the hydration coupling system may include a coupling device 614 that extends from the fluid system to the sealing mechanism 610 and/or 612. The coupling device 614 may provide a conduit to release hydrating fluid between the sealing members 613 and into contact with the flexible elongated device 602 that extends through the sealing mechanisms. In this example, separate coupling devices 614 may be used to introduce hydrating fluid into each of the sealing mechanisms 610, 612. In other examples, a single coupling device may introduce hydrating fluid into only one of the sealing mechanisms. The sealing members 613 of the sealing mechanisms 610, 612 may contain the hydrating fluid, preventing migration externally of the connector device 604. As the flexible elongated device 602 passes through the connector device 604, it may contact the hydrating fluid within the sealing mechanisms. The hydrating fluid may active a hydrophilic lubricious layer, seep into pores and reservoirs in the flexible elongated device, and/or otherwise contribute to the lubricity of the flexible elongated device 602 as it extends through the anatomic orifice device 606 and into the patient anatomy.

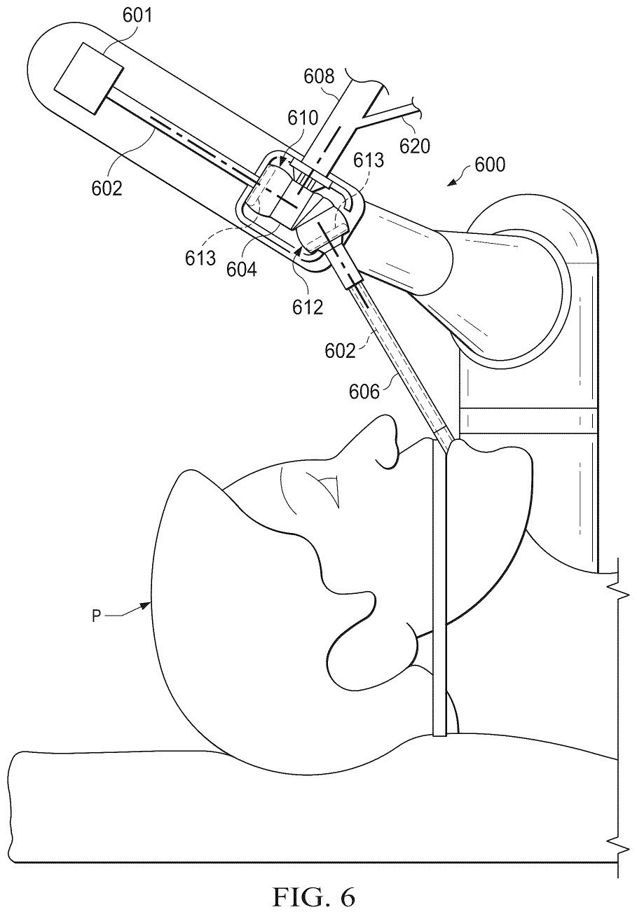

FIG. 6 illustrates the manipulator assembly 600 connected to the flexible elongated device 602. In this example, a hydration coupling system may include a coupling device 620 that connects the fluid system (e.g. the fluid system 112, 212) to the ventilation port 608. The coupling device 620 may provide a conduit to release hydrating fluid into the connector device 604 between the sealing mechanisms 610, 612 and into contact with the flexible elongated device 602 that extends through the sealing mechanisms. In this example, each sealing mechanism 610, 612 may include a single sealing member 613 or a plurality of sealing members. As the flexible elongated device 602 passes through the connector device 604, it may contact the hydrating fluid in the space between the sealing mechanisms. The hydrating fluid may active a hydrophilic lubricious layer, seep into pores and reservoirs in the flexible elongated device, and/or otherwise contribute to the lubricity of the flexible elongated device 602 as it extends through the anatomic orifice device 606 and into the patient anatomy.

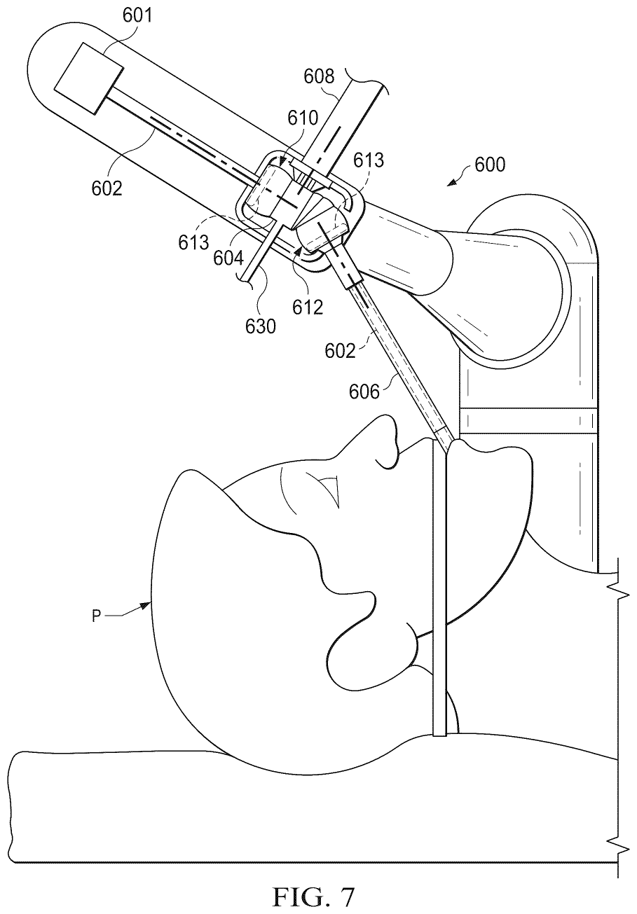

FIG. 7 illustrates the manipulator assembly 600 connected to the flexible elongated device 602. In this example, a hydration coupling system may include a coupling device 630 that connects the fluid system (e.g. the fluid system 112, 212) to the connector device 604, generally opposite the ventilation port 608. The coupling device 630 may provide a conduit to release hydrating fluid into the connector device 604 between the sealing mechanisms 610, 612 and into contact with the flexible elongated device 602 that extends through the sealing mechanisms. In this example, each sealing mechanism 610, 612 may include a single sealing member 613 or a plurality of sealing members. As the flexible elongated device 602 passes through the connector device 604, it may contact the hydrating fluid in the space between the sealing mechanisms. The hydrating fluid may active a hydrophilic lubricious layer, seep into pores and reservoirs in the flexible elongated device, and/or otherwise contribute to the lubricity of the flexible elongated device 602 as it extends through the anatomic orifice device 606 and into the patient anatomy.

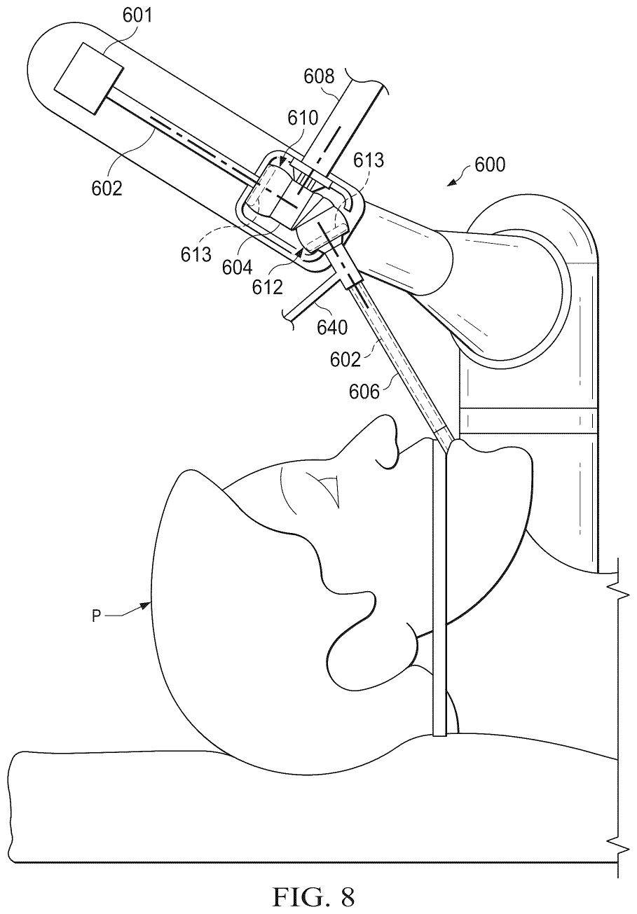

FIG. 8 illustrates the manipulator assembly 600 connected to the flexible elongated device 602. In this example, a hydration coupling system may include a coupling device 640 that connects the fluid system (e.g. the fluid system 112, 212) to the anatomic orifice device 606. The coupling device 640 may provide a conduit to release hydrating fluid into the anatomic orifice device 606, distally of the connector device 604, and into contact with the flexible elongated device 602 that extends through the anatomic orifice device 606. As the flexible elongated device 602 passes through anatomic orifice device 606, it may contact the hydrating fluid within the sealing mechanisms. The hydrating fluid may active a hydrophilic lubricious layer, seep into pores and reservoirs in the flexible elongated device, and/or otherwise contribute to the lubricity of the flexible elongated device 602 as it extends through the anatomic orifice device 606 and into the patient anatomy.

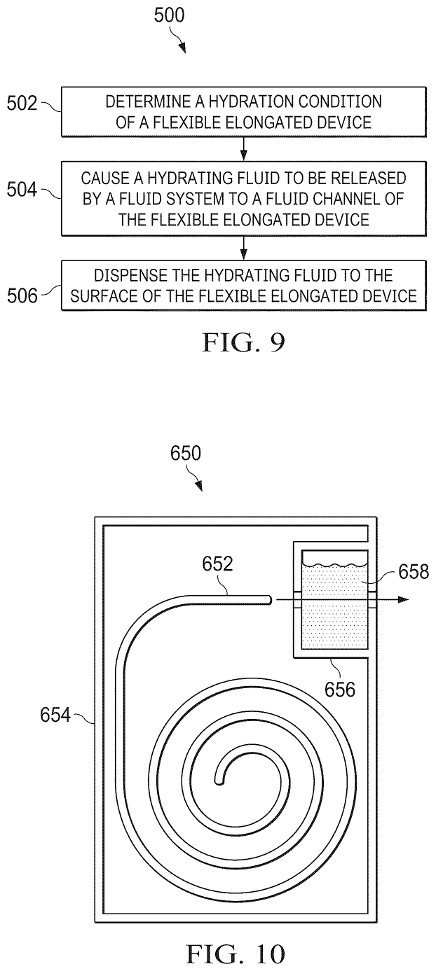

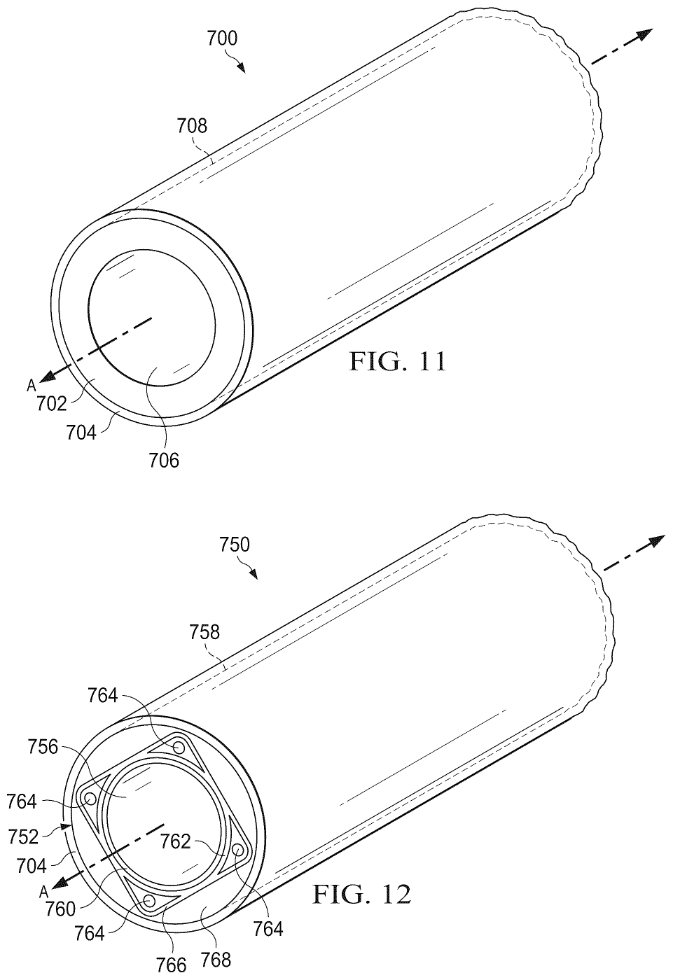

FIG. 9 is a flowchart illustrating a method 500 for hydrating a flexible elongated device. The method 500 is illustrated as a set of operations or processes that may be performed in the same or in a different order than the order shown. One or more of the illustrated processes may be omitted in some examples of the method. Additionally, one or more processes that are not expressly illustrated in FIG. 9 may be included before, after, in between, or as part of the illustrated processes. In some examples, one or more of the processes of method 500 may be implemented, at least in part, by a control system executing code stored on non-transitory, tangible, machine-readable media that when run by one or more processors (e.g., the processors of a control system 218, 1412) may cause the one or more processors to perform one or more of the processes.

At process 502, a hydration condition of a flexible elongated device (e.g. flexible elongated device 100, 150, 200, 250, 270, 300, 310, 320, 330, 330, 400, 602, 652) may be determined. For example, the control system may receive an indication that the lubricious layer of the flexible elongated device is in a dehydrated condition. A dehydrated condition may correspond to a hydration level of the flexible elongated device (or the lubricious layer) that is at or below a threshold hydration level (e.g. at or below the dehydrated threshold level). The indication may be received, for example, from a user input at a user input device of a master assembly (e.g. master assembly 806) or from a hydration detection system (e.g. hydration detection system 220). In some examples, the process 502 may be omitted.

At a process 504, a hydrating fluid may be released by a fluid system to a fluid channel of the flexible elongated device. For example, the control system may cause the fluid system 212 to release the hydrating fluid to the fluid channel in response to the determination of the dehydrated condition. In some examples, releasing the hydrating fluid may include activating the stimulation system 401 to release the hydrating fluid from fluid reservoirs in the flexible elongated device. In some examples, releasing the hydrating fluid may include activating the fluid system to release the hydrating fluid into a hydration coupling system external of the patient anatomy.

At a process 506, the hydrating fluid may be dispensed to the surface of the flexible elongated device. For example, the hydrating fluid may flow through the fluid channel, through one or more pores, and into or along the lubricious layer or along the surface of the flexible elongated body (if a lubricious layer is omitted). In other examples, the hydrating fluid, introduced through a hydration coupling system, may flow onto the surface of the flexible elongated device. In some examples, the hydrating fluid may be dispensed to the lubricious layer to transition the lubricious layer from a dehydrated condition to a hydrated condition. In some examples, the flow of the hydrating fluid may be terminated when the hydration level is above the dehydration threshold level or when a hydration threshold level is reached.

The lubricated flexible elongated device may help prevent stick-slip behaviors, irregular sliding, device buckling, or other behaviors that may impede navigation toward a target location in the patient anatomy. The method 500 may be performed while the flexible elongated device is inserted in a patient anatomy and thus may reduce or eliminate the need to withdraw the flexible elongated device from the patient anatomy to provide the re-hydration. This may be particularly useful for long medical procedures such as multiple biopsies or procedures that include both biopsy and treatment in a single event.

FIG. 10 illustrates a packaging hydration system 650 in which a flexible elongated device 652 (e.g. flexible elongated device 100, 150, 200, 250, 270, 300, 310, 320, 330, 330, 400, 602) may be packaged after manufacturing. The packaging hydration system 650 may include a container 654 in which the flexible elongated device may be contained to preserve sterilization and provide protection during transport. The packaging hydration system 650 may also include a fluid reservoir 656 that may be within or coupled to the container 654. The fluid reservoir 656 may be filled with a hydrating fluid 658 either before shipping or at a medical facility prior to use of the flexible elongated device 652. The flexible elongated device 652 may be drawn through the fluid reservoir 656 as it is extracted from the container 654. As the flexible elongated device 602 passes through the fluid reservoir 656, it may contact the hydrating fluid 658. The hydrating fluid 658 may active a hydrophilic lubricious layer, seep into pores and reservoirs in the flexible elongated device, and/or otherwise contribute to the lubricity of the flexible elongated device 652.

The instrument systems described herein may also include flexible elongated devices (e.g., catheters, bronchoscopes, or endoscopes) that include a lubricious layer. As flexible elongated devices navigate anatomic passages, they may experience friction with the inner wall of the passageways or with components of manipulator systems to which they may be attached. Friction may compromise control and navigation of the flexible elongated device through increased resistance, stick-slip behavior, prolapse of the device externally or internally of the patient, or an inability to reach a target location. Flexible elongated devices may be coated with a lubricious layer or coating, such as hydrophilic coating, to reduce or eliminate the issues associated with friction. In some types of anatomic passageways, such as cardiovascular or neurovascular passageways, naturally-present body fluids or mucous may activate hydrophilic coated flexible elongated devices to augment lubrication provided by the anatomy. In contrast, flexible elongated devices navigating through other types of anatomic passageways (e.g., lung passageways) may be exposed to flowing air that dries the flexible elongated devices and increases the coefficient of friction of the flexible elongated device. Hydrophilic coatings activated by non-anatomic sources of hydration may be used to increase lubrication in these drier anatomic environments.

FIG. 11 illustrates a flexible elongated device 700 including a flexible elongated body 702 with a lubricious layer 704 extending over at least a portion of the length of an outer surface 708 of the body 702. The flexible elongated body 702 may extend along a longitudinal axis A and define a lumen 706 through which, for example, tools may be inserted or fluids may be introduced or evacuated. In some examples, the flexible elongated device 700 may be a component of a robotically assisted medical instrument system or a manually-controlled medical instrument system that controls articulation and insertion/retraction of the flexible elongated device 700. An example of a medical instrument system including a flexible elongated device that is bendable and steerable in multiple degrees of freedom is described below in FIGS. 23A and 23B (e.g., system 1500).

The lubricious layer 704 may include, for example, a hydrophilic substance that may promote lubricity, thereby reducing or preventing stick-slip or irregular sliding behaviors as the flexible elongated device 700 is introduced into a patient anatomy. The lubricious layer 704 may be applied to the outer surface 708 of the flexible elongated body 702 for example by dip coating, spray-on application, wrap material application, wipe-on application, or as a tubular overlay. In some examples, a hydrophilic lubricious layer may have a hydrated or activated condition in which the hydrophilic material is hydrated and retentive of fluid or may have an anhydrous or inactivated condition in which the hydrophilic material is anhydrous or dehydrated. In some examples, the lubricious layer 704 may include a pigment or dye that imparts an identifying or detectable characteristic to the lubricious layer. The pigment may identify which portion of the flexible elongated device include the lubricious layer and thus will transition to a hydrated condition when hydrated. The pigment may also or alternatively indicate wear or delamination of the lubricious layer, signaling for example that the lubricious layer should be reapplied to the device discarded. In some examples, the pigment may be a visible pigment that imparts an identifying color. In other examples, the pigment may be infrared or fluorescent and may be detectable with corresponding detectors. In some alternative examples, the lubricious layer may include a hydrophobic material.

FIG. 2 illustrates a flexible elongated device 750 that may be substantially similar to the flexible elongated device 700, with differences as described. The flexible elongated device 750 includes a flexible elongated body 752 and the lubricious layer 704 extending over at least a portion of the length of the outer surface of the body 752. The flexible elongated device 750 may have a longitudinal axis A. The flexible elongated body 752 includes a flexible liner layer 760 defining a lumen 756 through which, for example, tools may be inserted or fluids may be introduced or evacuated. In some examples the liner layer 760 may be formed from a polymer material such as polytetrafluoroethylene (PTFE) or another fluoropolymer. The flexible elongated body 752 may also include a support layer 762 extending along at least a portion of the liner layer 760. The support layer 762 may include a woven material including for example, coiled polymer fibers, braided polymer fibers, coiled metal fibers, braided metal fibers or may include a non-woven material such as a laser cut hypo tube. A fiber may include metal fiber, polymer fiber, natural fiber, or any other type of synthetic or naturally occurring fiber. The flexible elongated body 752 may also include elongated control members 764 extending along the support layer 762 for controlling articulation and steering of the flexible elongated device 750. Although four control members are shown, in various examples, the number of control members may be greater than or less than four. The flexible elongated body 752 may also include a support layer 766 extending around the control members 764. The support layer 766 may include a woven material including for example, coiled polymer fibers, braided polymer fibers, coiled metal fibers, braided metal fibers or may include a non-woven material such as a laser cut hypo tube. The flexible elongated body 752 may also include a flexible elastomeric layer 768. In some examples, the flexible elastomeric layer 768 may include an elongated thermoplastic elastomer that extends around the support layer 766. The flexible elastomeric layer may, for example, be laminated to the support layer 766 by heat shrink. For example, a heat shrink tubing, such as a fluorinated ethylene propylene tubing, may be applied around the flexible elastomeric layer 768 and then heated. The heat shrink tubing may be removed after the flexible elastomeric layer 768 is laminated onto and into the support layer 766. The lubricious layer 704 may be applied to an outer surface 758 of the flexible elongated body 752 (e.g., the outer surface of the flexible elastomeric layer 768) for example by dip coating, spray-on application, wrap material application, wipe-on application, or as a tubular overlay. In some examples, a hydrophilic lubricious layer may have a hydrated condition in which the hydrophilic material is hydrated or may have an anhydrous condition in which the hydrophilic material is in an anhydrous or dehydrated condition. The lubricious layer in the hydrated condition may be thicker than the lubricious layer in the anhydrous condition resulting from expansion of the lubricious layer. For example, the lubricious layer in a highly or fully hydrated condition may have a thickness of approximately 20-40 μm which may be slightly thicker than the lubricious layer in the anhydrous condition having a thickness of approximately 2-5 μm. The lubricious layer in the hydrated condition may form a coating texture on an outer surface of the lubricious layer based on the surface texture of the flexible elongated body under the lubricious layer.

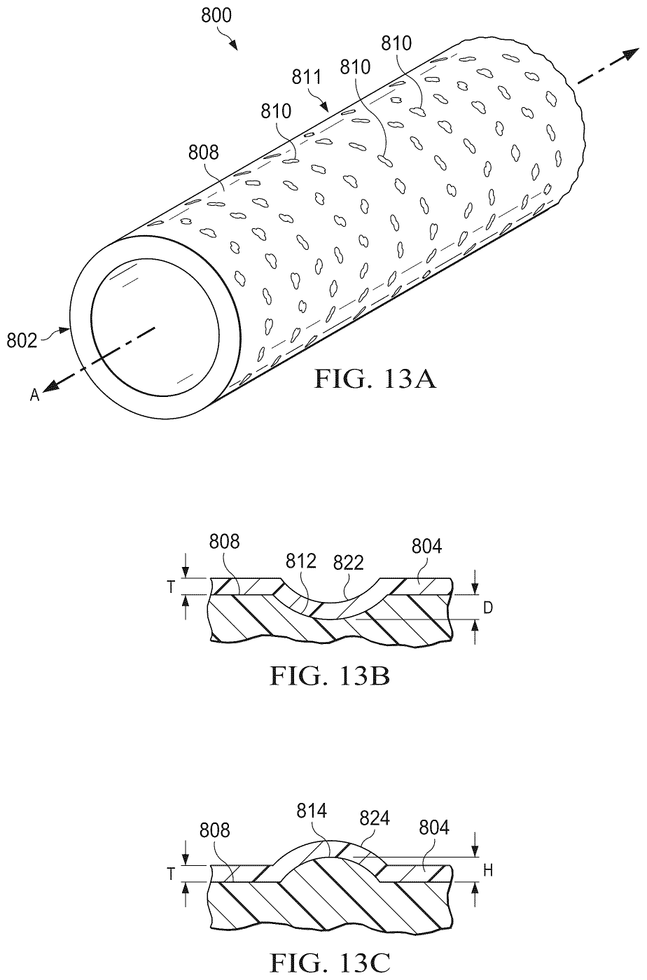

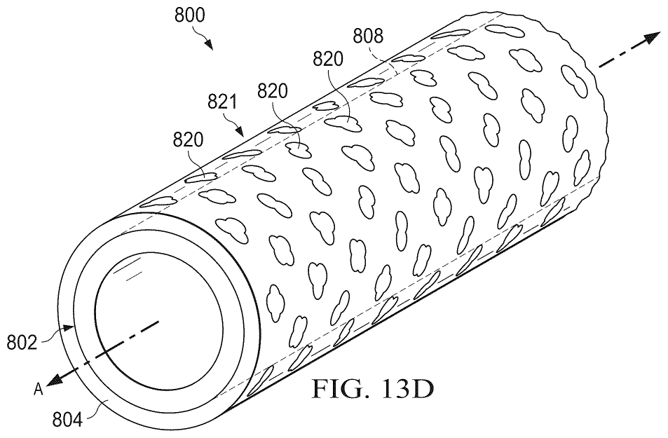

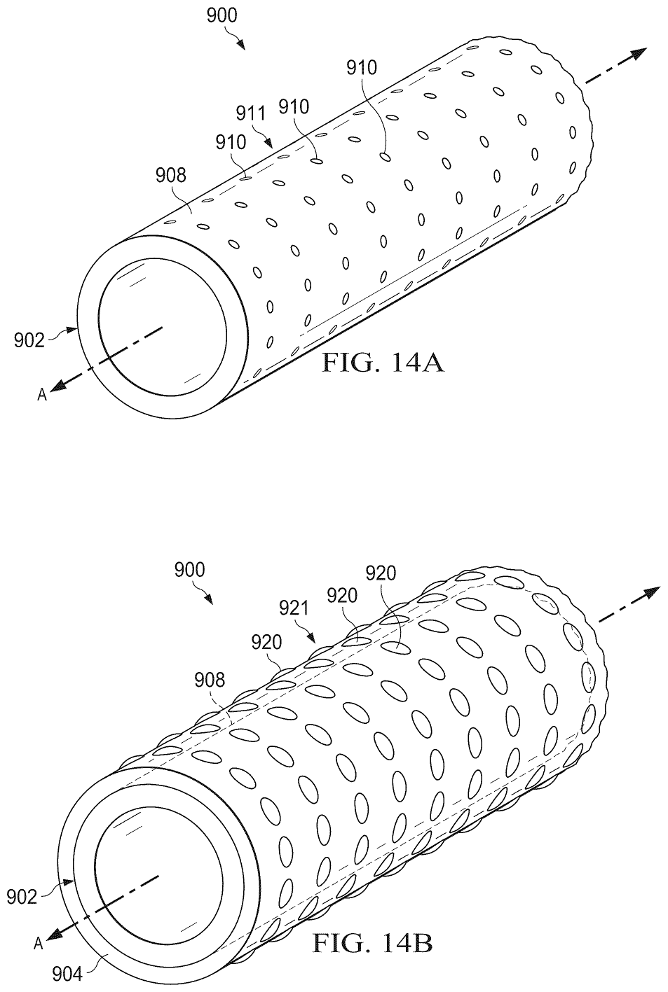

FIG. 13A illustrates a flexible elongated device 800 including a flexible elongated body 802 (e.g., the flexible elongated body 702, 752) extending along a longitudinal axis A. An outer surface 808 of the flexible elongated body 802 includes a plurality of microstructures 810 forming a surface texture 811. The plurality of microstructures may be irregularly arranged along the outer surface 808 or may be arranged in any of a variety of uniform patterns. In this example, the plurality of microstructures may be irregular in shape and/or may be unpatterned. In other examples described below, microstructures may have regular shapes and/or patterns. In some examples, the microstructures may be recessed microstructures 812 extending into the outer surface 808 of the flexible elongated body 802 as shown in FIG. 13B. For example, recessed microstructures may have a depth dimension between approximately 4 and 25 μm. In some examples, the microstructures may be raised microstructures 814 extending above the outer surface 808 of the flexible elongated body 802 as shown in FIG. 13C. For example, raised microstructures may have a height dimension between approximately 4 and 25 μm. In some examples, the plurality of microstructures 810 may include both recessed microstructures 812 and raised microstructures 814. As shown in FIG. 3D, a lubricious layer 804 (e.g. lubricious layer 704) may extend over at least a portion of the length of the outer surface 808. The lubricious layer 804 in the hydrated condition may form a coating texture 821 on an outer surface of the lubricious layer 804 comprising contoured features 820 based on the surface texture 811 or following the underlying microstructures 810 of the surface texture. For example and as shown in FIG. 13B, the thin lubricious layer 804 may extend into recessed microstructures 812 without completely filling the recess. The recesses microstructure 812 may have a depth dimension D, and the hydrated lubricious layer 804 may have the thickness dimension T. Thus, the recessed microstructures 812 may cause a pocket or crevice formation 822 in the lubricious layer 804. For example and as shown in FIG. 13C, the lubricious layer 804 may cover the raised microstructures 814. Thus, the raised microstructures 814 may cause a mound or bump formation 824 of the lubricious layer 804. The raised microstructure may have a height dimension H, and the hydrated lubricious layer 804 may have the thickness dimension T. The contoured features 820, formed or exhibited by the lubricious layer 804 overlaying the raised microstructures 814, may include raised features, recessed features, or a combination of recessed and raised features. With the lubricious layer 804 in the hydrated condition as shown in FIG. 13D, the corrugation of the surface texture 811 may remain in the coating texture 821 depending on the ratio of pitch of feature size of the microstructures 810 compared to thickness of the lubricious layer 804 in the hydrated condition. This allows the lubricious layer to conform to features rather than be a smooth surface. In some examples, the microstructure size dimension (e.g. depth D or height H in FIG. 13B/13C) may be approximately 1.5 times the dimension of the hydrated thickness (e.g. thickness T in FIG. 13B/13C) of the lubricious layer. For example, a microstructure size dimension of 40-70 μm may be approximately 1.5 times a hydrated thickness dimension of 20-40 μm. In some examples, the microstructure dimension may be approximately 2-10 times the dimension of the hydrated thickness of the lubricious layer.

The lubricious layer 804 may include, for example, a hydrophilic or a hydrophobic substance that may promote lubricity thereby reducing or preventing stick-slip or irregular sliding behaviors as the flexible elongated device 800 is introduced into a patient anatomy. The lubricious layer 804 may be applied to an outer surface 708 of the flexible elongated body 702 for example by dip coating, spray-on application, wrap material application, wipe-on application, or as a tubular overlay.

A hydrophilic lubricious layer 804 may have a hydrated condition in which the hydrophilic material is hydrated (e.g., by water, saline, human anatomic fluid/mucous, or another hydrating liquid) or may have an anhydrous condition in which the hydrophilic material is in an anhydrous or dehydrated condition. The lubricious layer 804 in the hydrated condition may be thicker than the lubricious layer in the anhydrous condition resulting from expansion of the lubricious layer. For example, in the hydrated condition the hydrophilic lubricious layer may be expanded approximately 5-10 times the thickness of the anhydrous condition. For example, a hydrophilic lubricious layer with a thickness of between approximately 2 and 3 μm may expand to a thickness of approximately 20 μm in the hydrated condition. Recessed microstructures 812 that have a depth greater than approximately 1.5 times the thickness of the lubricious layer in the hydrated condition will allow the lubricious layer to form the contoured features 820 that follow or maintain the underlying texture when in the hydrated condition. Similarly, raised microstructures spaced apart from other raised microstructures by interstitial spaces with a depth greater than approximately 1.5 times the thickness of the lubricious layer in the hydrated condition will allow the lubricious layer to form the contoured features 820 that follow or maintain the underlying texture when in the hydrated condition.

The textured surface formed by the microstructures 810 and the corresponding contoured features 820 when the hydrophilic lubricious layer 804 in in the hydrated condition may promote lubricity and reduce friction in several ways. For example, the hydrophilic lubricious layer in the hydrated condition may have a coefficient of friction with anatomic tissue that is lower than a coefficient of friction of the uncoated outer surface 808 of the flexible elongated body 802. Additionally or alternatively, contoured features 820 may form reservoirs, trapping fluid that may further enhance the lubricity of the flexible elongated device 800. Additionally or alternatively, the discontinuous coating texture formed by the contoured features 820 may reduce the surface area of the flexible elongated device 800 that contacts the anatomic passageways, thus reducing contact friction force.

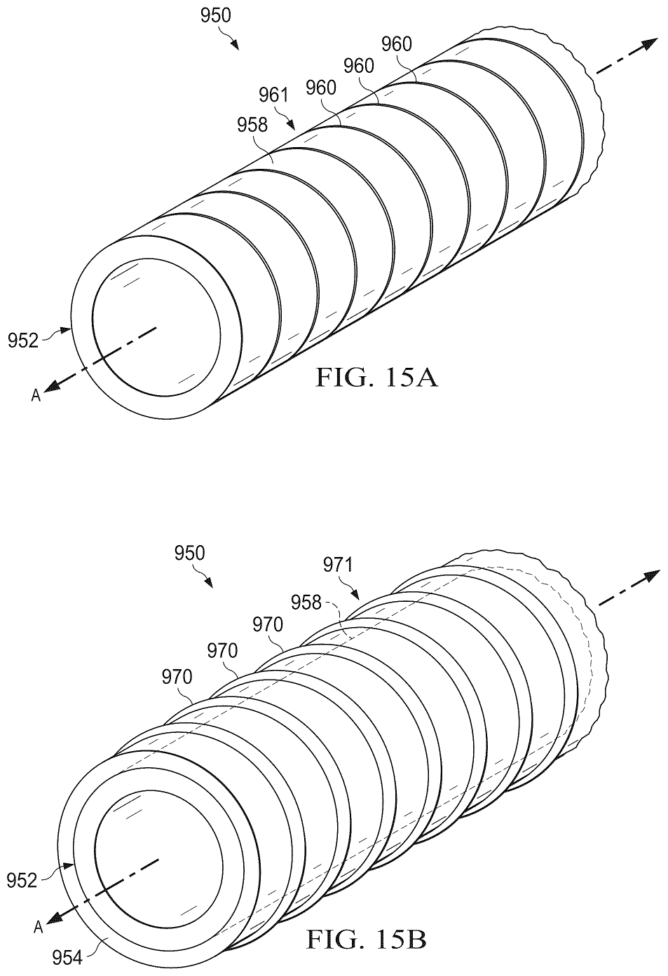

FIG. 14A illustrates a flexible elongated device 900 including a flexible elongated body 902 (e.g., the flexible elongated body 702, 752). An outer surface 908 of the flexible elongated body 902 includes a plurality of microstructures 910 forming a surface texture 911. In this example, the plurality of microstructures may be generally uniform in shape and may be in a patterned arrangement such as an array or grid with approximately equidistant spacing between the microstructures 910. For example, a distance between approximately 10 and 100 μm may extend between adjacent microstructures. In some examples, the microstructures may be recessed microstructures, raised microstructures, or a combination of types of microstructures. As shown in FIG. 14B, a lubricious layer 904 (e.g. lubricious layer 704) may extend over at least a portion of the length of the outer surface 908. The lubricious layer 904 may form a coating texture 921 comprising contoured features 920 based on or following the underlying microstructures 910 of the surface texture. The contoured features 920 may include raised features, recessed features, or a combination of recessed and raised features. When the hydrophilic lubricious layer 904 in the hydrated condition, the textured surface formed by the microstructures 910 and the corresponding contoured features 920 may promote lubricity and reduce friction as previously described.

FIG. 15A illustrates a flexible elongated device 950 including a flexible elongated body 952 (e.g., the flexible elongated body 702, 752). An outer surface 958 of the flexible elongated body 952 includes a plurality of microstructures 960 forming a surface texture 961. In this example, the plurality of microstructures may be circumferential rings arranged generally perpendicular to a longitudinal axis A of the body 952. The ring microstructures 960 may have approximately equidistant spacing. In some examples, the microstructures may be recessed microstructures, raised microstructures, or a combination of types of microstructures. As shown in FIG. 15B, a lubricious layer 954 (e.g. lubricious layer 704) may extend over at least a portion of the length of the outer surface 958. The lubricious layer 954 may form a coating texture 971 comprising contoured features 970 based on or following the underlying microstructures 960 of the surface texture. The contoured features 970 may include raised features, recessed features, or a combination of recessed and raised features. When the hydrophilic lubricious layer 954 in the hydrated condition, the textured surface formed by the microstructures 960 and the corresponding contoured features 970 may promote lubricity and reduce friction as previously described.

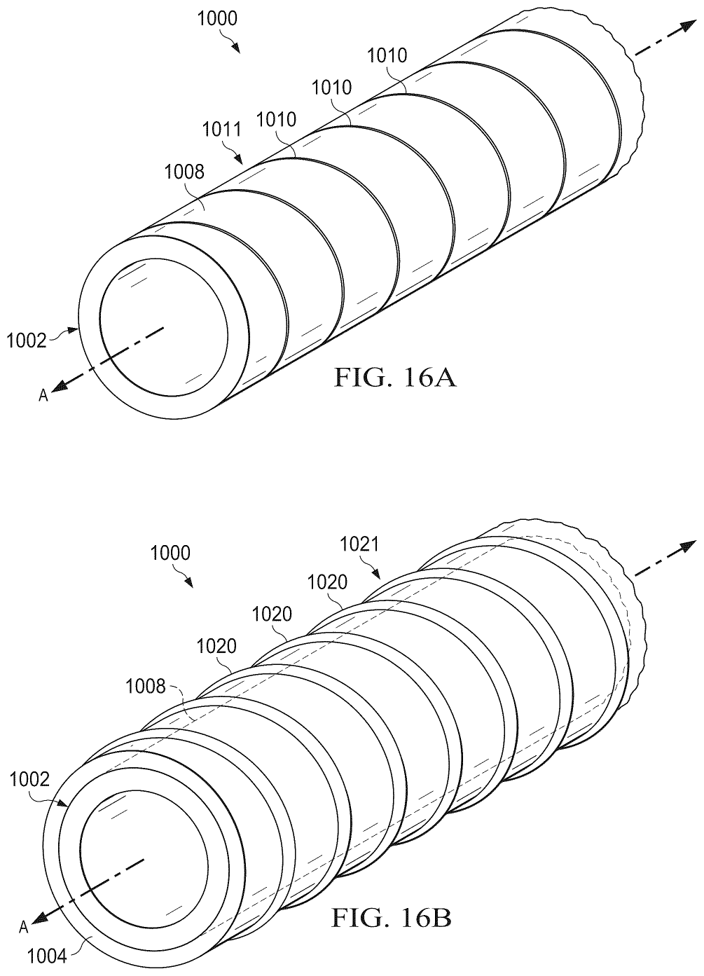

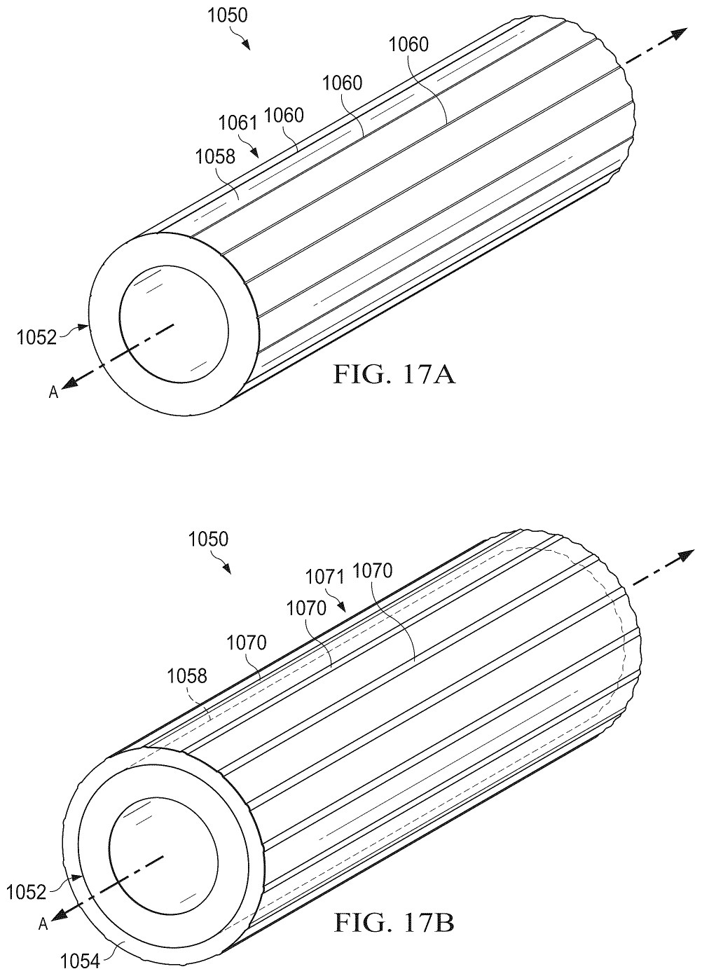

FIG. 16A illustrates a flexible elongated device 1000 including a flexible elongated body 1002 (e.g., the flexible elongated body 702, 752). An outer surface 1008 of the flexible elongated body 1002 includes a plurality of microstructures 1010 forming a surface texture 1011. In this example, the plurality of microstructures may be spiral or helical microstructures extending around the longitudinal axis A of the body 1002. The spiral microstructures 1010 may have approximately equidistant spacing. In some examples, the spiral microstructures may include dual or triple helix configurations with two, three, or more intertwined spiral microstructures. In some examples, the microstructures may be recessed microstructures, raised microstructures, or a combination of types of microstructures. As shown in FIG. 16B, a lubricious layer 1004 (e.g. lubricious layer 704) may extend over at least a portion of the length of the outer surface 1008. The lubricious layer 1004 may form a coating texture 1021 comprising contoured features 1020 based on or following the underlying microstructures 1010 of the surface texture. The contoured features 1020 may include raised features, recessed features, or a combination of recessed and raised features. When the hydrophilic lubricious layer 1004 is in the hydrated condition, the textured surface formed by the microstructures 1010 and the corresponding contoured features 1020 may promote lubricity and reduce friction as previously described.

FIG. 7A illustrates a flexible elongated device 1050 including a flexible elongated body 1052 (e.g., the flexible elongated body 702, 752). An outer surface 1058 of the flexible elongated body 1052 includes a plurality of microstructures 1060 forming a surface texture 1061. In this example, the plurality of microstructures may be radial corrugations extending generally parallel to and radially distributed around the longitudinal axis A of the body 1052. The radial microstructures 1060 may have approximately equidistant spacing. In some examples, the microstructures may be recessed microstructures, raised microstructures, or a combination of types of microstructures. As shown in FIG. 17B, a lubricious layer 1054 (e.g. lubricious layer 704) may extend over at least a portion of the length of the outer surface 1058. The lubricious layer 1054 may form a coating texture 1071 comprising contoured features 1070 based on or following the underlying microstructures 1060 of the surface texture. The contoured features 1070 may include raised features, recessed features, or a combination of recessed and raised features. When the hydrophilic lubricious layer 1054 is in the hydrated condition, the textured surface formed by the microstructures 1060 and the corresponding contoured features 1070 may promote lubricity and reduce friction as previously described.

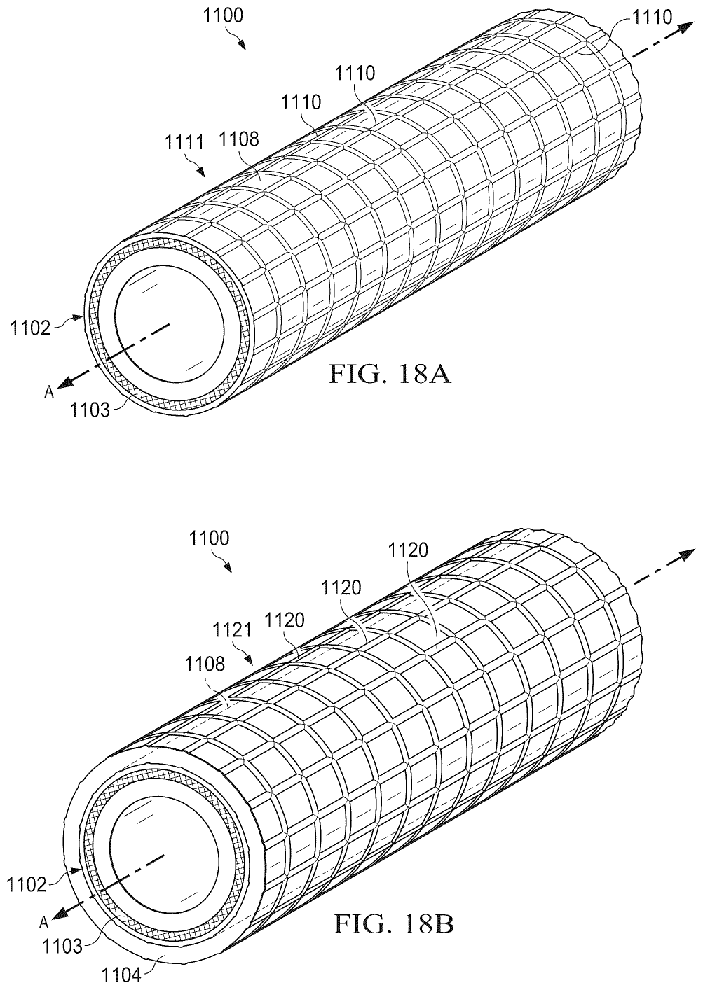

FIG. 18A illustrates a flexible elongated device 1100 including a flexible elongated body 1102 (e.g., the flexible elongated body 702, 752). The flexible elongated body 1102 may include a support layer 1103 extending beneath an outer surface 1108 of the flexible elongated body 1102. The support layer 1103 may include a woven material including for example, coiled polymer fibers, braided polymer fibers, coiled metal fibers, braided metal fibers or may include a non-woven material such as a laser cut hypo tube.

The underlying support layer 1103 imparts a plurality of microstructures 1110, which form a surface texture 1111, to the outer surface 1108 of the flexible elongated body 1102. In this example, the plurality of microstructures 1110 may be a woven pattern of microstructures extending along the outer surface 1108 and along the longitudinal axis A of the body 1102. The microstructures may include a combination of recessed and raised microstructures. As shown in FIG. 18B, a lubricious layer 1104 (e.g. lubricious layer 704) may extend over at least a portion of the length of the outer surface 1108. The lubricious layer 1104 may form a coating texture 1121 comprising contoured features 1120 based on or following the underlying microstructures 1110. The contoured features 1120 may include raised features, recessed features, or a combination of recessed and raised features. When the hydrophilic lubricious layer 1104 is in the hydrated condition, the textured surface formed by the microstructures 1110 and the corresponding contoured features 1120 may promote lubricity and reduce friction as previously described.

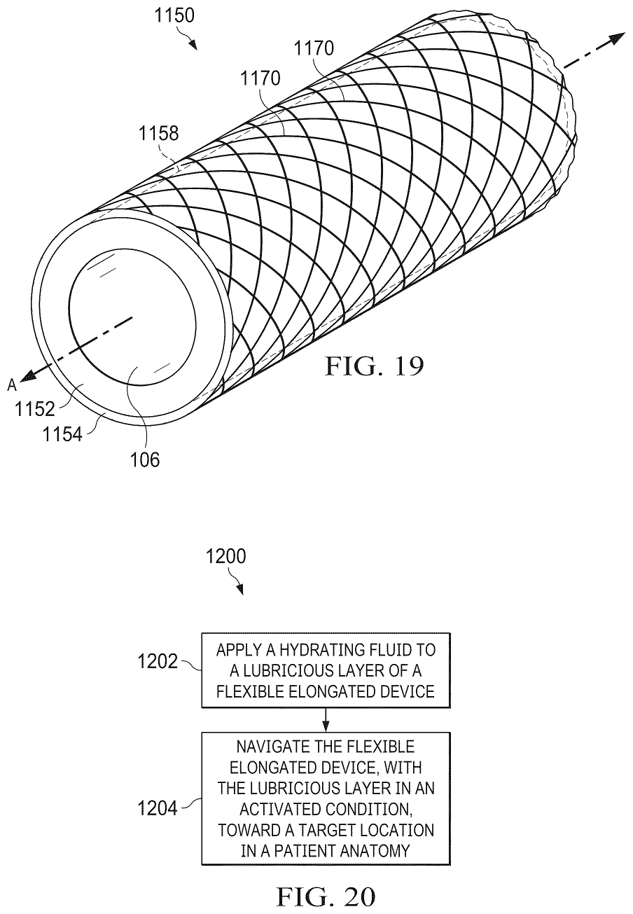

FIG. 19 illustrates a flexible elongated device 1150 including a flexible elongated body 1152 (e.g., the flexible elongated body 702, 752) with an outer surface 1158. A lubricious layer 1154 (e.g. lubricious layer 704) may extend over at least a portion of the length of the outer surface 1158. In this example, contoured features 1170 may be formed in the lubricious layer 1154 after the lubricious layer is deposited on the outer surface 1158. For example, the contoured features 1170 may be laser etched, micromachined, or otherwise engraved into the lubricious layer. In other examples, the contoured features may be imprinted to the lubricious layer by a lamination layer or tool that may be applied to the lubricious layer, optionally with heat, and then removed. The contoured features 1170 may include raised and recessed contoured features. In this example the outer surface 1158 may be smooth or may further include microstructures that impart additional contoured features to the lubricous layer as previously described. When the hydrophilic lubricious layer 1154 is in the hydrated condition, the contoured features 1170 may promote lubricity and reduce friction as previously described.

FIG. 20 is a flowchart illustrating a method 1200 for utilizing a utilizing a flexible elongated device including a lubricious layer. The method 1200 is illustrated as a set of operations or processes that may be performed in the same or in a different order than the order shown. One or more of the illustrated processes may be omitted in some examples of the method. Additionally, one or more processes that are not expressly illustrated in FIG. 20 may be included before, after, in between, or as part of the illustrated processes. In some examples, one or more of the processes of method 1200 may be implemented, at least in part, by a control system executing code stored on non-transitory, tangible, machine-readable media that when run by one or more processors (e.g., the processors of a control system 1412) may cause the one or more processors to perform one or more of the processes.

At process 1202, a flexible elongated device (e.g. flexible elongated device 700, 750, 800, 900, 950, 1000, 1050, 1100, 1150) including a hydrophilic lubricious layer may be transitioned to a hydrated state by applying a hydrating liquid to the lubricious layer. The hydrating liquid may be applied before the flexible elongated device is inserted into a patient anatomy or while the flexible elongated device is in the patient anatomy. The hydrating liquid may be, for example, water, saline, or a human anatomic fluid or mucous. Contoured features of the lubricious layer, formed for example by microstructures on an underlying flexible elongated body or by microstructures in the lubricious layer formed after the lubricious layer is applied, may lower insertion force or frictional force. For example, the hydrophilic lubricious layer in the hydrated condition may have a reduced coefficient of friction with anatomic tissue as compared to an uncoated outer surface of the flexible elongated body. The contoured features may form reservoirs, trapping fluid that may further enhance the lubricity of the flexible elongated device. Additionally or alternatively, the discontinuous texture formed by the contoured features may reduce the surface area of the flexible elongated device that contacts the anatomic passageways, thus reducing contact friction force.

At a process 1204, the flexible elongated device, with the hydrophilic lubricious layer in a hydrated condition, may be navigated toward a target location in a patient anatomy. In some examples, the flexible elongated device may be hand-held or otherwise manually controlled during navigation. In other examples, the flexible elongated device may be controlled by a robot-assisted medical system. In some examples, a robot-assisted medical system may sense that the flexible elongated device has remained static in an airway longer than a predetermined duration or is otherwise imprecisely responsive to commanded motion, due to, for example, the relatively dry environment of a lung airway. Responsive to the detected condition, a robot assisted medical system may initiate a micro motion such as a small retraction or a spiral motion to break any static friction and reactivate the hydrophilic lubricious layer by touching nearby mucous or fluid before further advancing. The hydrophilic lubricious layer and contoured features formed by the lubricious layer may help prevent stick-slip behaviors, irregular sliding, device buckling, or other behaviors that may impede navigation toward the target location.

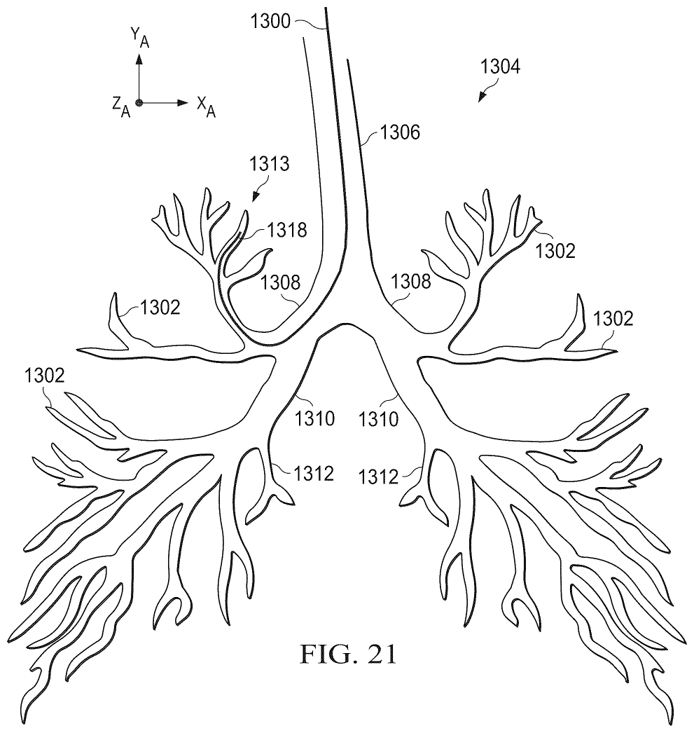

FIG. 21 illustrates a medical instrument system 1300 including a flexible elongated device (e.g. flexible elongated device 100, 150, 200, 250, 270, 300, 310, 320, 330, 330, 400, 602, 700, 750, 800, 900, 950, 1000, 1050, 1100, 1150) extending within branched anatomic passageways or airways 1302 of an anatomical structure 1304. In some examples the anatomic structure 1304 may be a lung and the passageways 1302 may include the trachea 1306, primary bronchi 1308, secondary bronchi 1310, and tertiary bronchi 1312. The anatomic structure 1304 has an anatomical frame of reference (X_{A}, Y_{A}, Z_{A}). A distal end portion 1318 of the medical instrument system 1300 may be advanced into an anatomic opening (e.g., a patient mouth) and through the anatomic passageways 1302 to perform a medical procedure, such as a biopsy, ablation, electroporation, or other type of diagnostic or therapeutic procedure, at or near a target tissue 1313. The medical instrument system 1300 may be suitable for use in, for example, surgical, diagnostic (e.g., biopsy), or therapeutic (e.g., ablation, electroporation, etc.) procedures. While some embodiments are provided herein with respect to such procedures, any reference to medical or surgical instruments and medical or surgical methods is non-limiting. The systems, instruments, and methods described herein may be used for animals, human cadavers, animal cadavers, portions of human or animal anatomy, non-surgical diagnosis, as well as for industrial systems, general or special purpose robotic systems, general or special purpose robot-assisted medical systems.

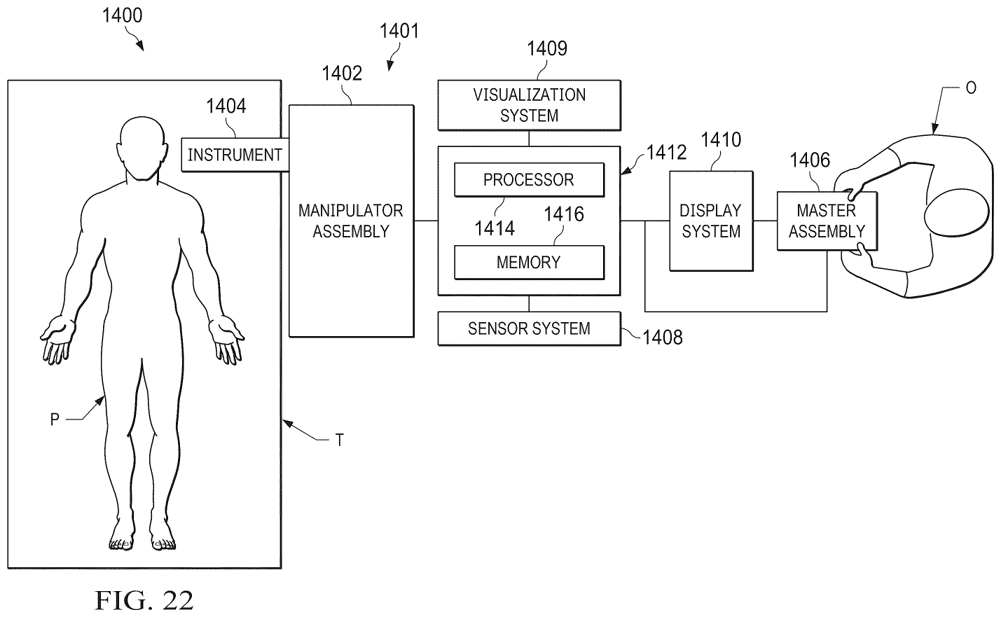

FIG. 22 illustrates a medical system 1400 that may include a manipulator assembly 1402 that controls the operation of a medical instrument 1404 (e.g., medical instrument system 1300) in performing various procedures on a patient P. Medical instrument 1404 may extend into an internal site within the body of patient P via an opening in the body of patient P. The manipulator assembly 1402 may be robot-assisted, non-assisted, or a hybrid robot-assisted and non-assisted assembly with select degrees of freedom of motion that may be motorized and/or robot-assisted and select degrees of freedom of motion that may be non-motorized and/or non-assisted. The manipulator assembly 1402 may be mounted to and/or positioned near a patient table T. A master assembly 1406 allows an operator O (e.g., a surgeon, a clinician, a physician, or other user) to control the manipulator assembly 1402. In some examples, the master assembly 1406 allows the operator O to view the procedural site or other graphical or informational displays. In some examples, the manipulator assembly 1402 may be excluded from the medical system 1400 and the instrument 1404 may be controlled directly by the operator O. In some examples, the manipulator assembly 1402 may be manually controlled by the operator O. Direct operator control may include various handles and operator interfaces for hand-held operation of the instrument 1404.

The master assembly 1406 may be located at a surgeon's console which is in proximity to (e.g., in the same room as) a patient table T on which patient P is located, such as at the side of the patient table T. In some examples, the master assembly 1406 is remote from the patient table T, such as in in a different room or a different building from the patient table T. The master assembly 1406 may include one or more control devices for controlling the manipulator assembly 1402. The control devices may include any number of a variety of input devices, such as joysticks, trackballs, scroll wheels, directional pads, buttons, data gloves, trigger-guns, hand-operated controllers, voice recognition devices, motion or presence sensors, and/or the like.

The manipulator assembly 1402 supports the medical instrument 1404 and may include a kinematic structure of links that provide a set-up structure. The links may include one or more non-servo controlled links (e.g., one or more links that may be manually positioned and locked in place) and/or one or more servo controlled links (e.g., one or more links that may be controlled in response to commands, such as from a control system 1412). The manipulator assembly 1402 may include a plurality of actuators (e.g., motors) that drive inputs on the medical instrument 1404 in response to commands, such as from the control system 1412. The actuators may include drive systems that move the medical instrument 1404 in various ways when coupled to the medical instrument 1404. For example, one or more actuators may advance medical instrument 1404 into a naturally or surgically created anatomic orifice. Actuators may control articulation of the medical instrument 1404, such as by moving the distal end (or any other portion) of medical instrument 1404 in multiple degrees of freedom. These degrees of freedom may include three degrees of linear motion (e.g., linear motion along the X, Y, Z Cartesian axes) and in three degrees of rotational motion (e.g., rotation about the X, Y, Z Cartesian axes). One or more actuators may control rotation of the medical instrument about a longitudinal axis. Actuators can also be used to move an articulable end effector of medical instrument 1404, such as for grasping tissue in the jaws of a biopsy device and/or the like or may be used to move or otherwise control tools (e.g., imaging tools, ablation tools, biopsy tools, electroporation tools, etc.) that are inserted within the medical instrument 1404.

The medical system 1400 may include a sensor system 1408 with one or more sub-systems for receiving information about the manipulator assembly 1402 and/or the medical instrument 1404. Such sub-systems may include a position sensor system (e.g., that uses electromagnetic (EM) sensors or other types of sensors that detect position or location); a shape sensor system for determining the position, orientation, speed, velocity, pose, and/or shape of a distal end and/or of one or more segments along a flexible body of the medical instrument 1404; a visualization system 1409 (e.g., using a color imaging device, an infrared imaging device, an ultrasound imaging device, an x-ray imaging device, a fluoroscopic imaging device, a computed tomography (CT) imaging device, a magnetic resonance imaging (MRI) imaging device, or some other type of imaging device) for capturing images, such as from the distal end of medical instrument 1404 or from some other location; and/or actuator position sensors such as resolvers, encoders, potentiometers, and the like that describe the rotation and/or orientation of the actuators controlling the medical instrument 1404.

The medical system 1400 may include a display system 1410 for displaying an image or representation of the procedural site and the medical instrument 1404. Display system 1410 and master assembly 1406 may be oriented so physician O can control medical instrument 1404 and master assembly 1406 with the perception of telepresence.

In some embodiments, the medical instrument 1404 may include a visualization system 1409, which may include an image capture assembly that records a concurrent or real-time image of a procedural site and provides the image to the operator O through one or more displays of display system 1410. The image capture assembly may include various types of imaging devices. The concurrent image may be, for example, a two-dimensional image or a three-dimensional image captured by an endoscope positioned within the anatomical procedural site. In some examples, the visualization system may include endoscopic components that may be integrally or removably coupled to medical instrument 1404. Additionally or alternatively, a separate endoscope, attached to a separate manipulator assembly, may be used with medical instrument 1404 to image the procedural site. The visualization system may be implemented as hardware, firmware, software or a combination thereof which interact with or are otherwise executed by one or more computer processors, such as of the control system 1412.

Display system 1410 may also display an image of the procedural site and medical instruments, which may be captured by the visualization system. In some examples, the medical system 1400 provides a perception of telepresence to the operator O. For example, images captured by an imaging device at a distal portion of the medical instrument 1404 may be presented by the display system 1410 to provide the perception of being at the distal portion of the medical instrument 1404 to the operator O. The input to the master assembly 1406 provided by the operator O may move the distal portion of the medical instrument 1404 in a manner that corresponds with the nature of the input (e.g., distal tip turns right when a trackball is rolled to the right) and results in corresponding change to the perspective of the images captured by the imaging device at the distal portion of the medical instrument 1404. As such, the perception of telepresence for the operator O is maintained as the medical instrument 1404 is moved using the master assembly 1406. The operator O can manipulate the medical instrument 1404 and hand controls of the master assembly 1406 as if viewing the workspace in substantially true presence, simulating the experience of an operator that is physically manipulating the medical instrument 1404 from within the patient anatomy.

In some examples, the display system 1410 may present virtual images of a procedural site that are created using image data recorded pre-operatively or intra-operatively, such as image data created using computed tomography (CT), magnetic resonance imaging (MRI), positron emission tomography (PET), fluoroscopy, thermography, ultrasound, optical coherence tomography (OCT), thermal imaging, impedance imaging, laser imaging, nanotube X-ray imaging, and/or the like. The virtual images may include two-dimensional, three-dimensional, or higher-dimensional (e.g., including, for example, time based or velocity-based information) images. In some examples, one or more models are created from pre-operative or intra-operative image data sets and the virtual images are generated using the one or more models.

In some examples, for purposes of imaged guided medical procedures, display system 1410 may display a virtual image that is generated based on tracking the location of medical instrument 1404. For example, the tracked location of the medical instrument 1404 may be registered (e.g., dynamically referenced) with the model generated using the pre-operative or intra-operative images, with different portions of the model correspond with different locations of the patient anatomy. As the medical instrument 1404 moves through the patient anatomy, the registration is used to determine portions of the model corresponding with the location and/or perspective of the medical instrument 1404 and virtual images are generated using the determined portions of the model. This may be done to present the operator O with virtual images of the internal procedural site from viewpoints of medical instrument 1404 that correspond with the tracked locations of the medical instrument 1404.

The medical system 1400 may also include the control system 1412, which may include processing circuitry that implements the some or all of the methods or functionality discussed herein. The control system 1412 may include at least one memory 1416 and at least one processor 1414 for controlling the operations of the manipulator assembly 1402, the medical instrument 1404, the master assembly 1406, the sensor system 1408, and/or the display system 1410. Control system 1412 may include instructions (e.g., a non-transitory machine-readable medium storing the instructions) that when executed by the at least one processor, configures the one or more processors to implement some or all of the methods or functionality discussed herein. While the control system 1412 is shown as a single block in FIG. 22, the control system 1412 may include two or more separate data processing circuits with one portion of the processing being performed at the manipulator assembly 1402, another portion of the processing being performed at the master assembly 1406, and/or the like. In some examples, the control system 1412 may include other types of processing circuitry, such as application-specific integrated circuits (ASICs) and/or field-programmable gate array (FPGAs). The control system 1412 may be implemented using hardware, firmware, software, or a combination thereof.

In some examples, the control system 1412 may receive feedback from the medical instrument 1404, such as force and/or torque feedback. Responsive to the feedback, the control system 1412 may transmit signals to the master assembly 1406. In some examples, the control system 1412 may transmit signals instructing one or more actuators of the manipulator assembly 1402 to move the medical instrument 1404. In some examples, the control system 1412 may transmit informational displays regarding the feedback to the display system 1410 for presentation or perform other types of actions based on the feedback.

The control system 1412 may include a virtual visualization system to provide navigation assistance to operator O when controlling the medical instrument 1404 during an image-guided medical procedure. Virtual navigation using the virtual visualization system may be based upon an acquired pre-operative or intra-operative dataset of anatomic passageways of the patient P. The control system 1412 or a separate computing device may convert the recorded images, using programmed instructions alone or in combination with operator inputs, into a model of the patient anatomy. The model may include a segmented two-dimensional or three-dimensional composite representation of a partial or an entire anatomic organ or anatomic region. An image data set may be associated with the composite representation. The virtual visualization system may obtain sensor data from the sensor system 1408 that is used to compute an (e.g., approximate) location of the medical instrument 1404 with respect to the anatomy of patient P. The sensor system 1408 may be used to register and display the medical instrument 1404 together with the pre-operatively or intra-operatively recorded images. For example, PCT Publication WO 2016/191298 (published Dec. 1, 2016 and titled “Systems and Methods of Registration for Image Guided Surgery”), which is incorporated by reference herein in its entirety, discloses example systems.

During a virtual navigation procedure, the sensor system 1408 may be used to compute the (e.g., approximate) location of the medical instrument 1404 with respect to the anatomy of patient P. The location can be used to produce both macro-level (e.g., external) tracking images of the anatomy of patient P and virtual internal images of the anatomy of patient P. The system may include one or more electromagnetic (EM) sensors, fiber optic sensors, and/or other sensors to register and display a medical instrument together with pre-operatively recorded medical images. For example, U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety, discloses example systems.

Medical system 1400 may further include operations and support systems (not shown) such as illumination systems, steering control systems, irrigation systems, and/or suction systems. In some embodiments, the medical system 1400 may include more than one manipulator assembly and/or more than one master assembly. The exact number of manipulator assemblies may depend on the medical procedure and space constraints within the procedural room, among other factors. Multiple master assemblies may be co-located or they may be positioned in separate locations. Multiple master assemblies may allow more than one operator to control one or more manipulator assemblies in various combinations.

FIG. 23A is a simplified diagram of a medical instrument system 1500 according to some embodiments. The medical instrument system 1500 includes a flexible elongated device 1502 (e.g. flexible elongated device 100, 150, 200, 250, 270, 300, 310, 320, 330, 330, 400, 602, 700, 750, 800, 900, 950, 1000, 1050, 1100, 1150), a drive unit 1504, and a medical tool 1526 that collectively is an example of a medical instrument 1404 of a medical system 1400. The medical system 1400 may be a robot-assisted system, a non-robot-assisted system, or a hybrid robot-assisted and non-assisted system, as explained with reference to FIG. 22. A visualization system 1531, tracking system 1530, and navigation system 1532 are also shown in FIG. 23A and are example components of the control system 1412 of the medical system 1400. In some examples, the medical instrument system 1500 may be used for non-robot-assisted exploratory procedures or in procedures involving traditional manually operated medical instruments, such as endoscopy. The medical instrument system 1500 may be used to gather (e.g., measure) a set of data points corresponding to locations within anatomic passageways of a patient, such as patient P.

The elongated device 1502 is coupled to the drive unit 1504. The elongated device 1502 includes a channel or lumen 1521 through which the medical tool 1526 may be inserted. The elongated device 1502 navigates within patient anatomy to deliver the medical tool 1526 to a procedural site. The elongated device 1502 includes a flexible body 1516 having a proximal end 1517 and a distal end 1518. In some examples, the flexible body 1516 may have an approximately 3 mm outer diameter. Other flexible body outer diameters may be larger or smaller.

Medical instrument system 1500 may include the tracking system 1530 for determining the position, orientation, speed, velocity, pose, and/or shape of the flexible body 1516 at the distal end 1518 and/or of one or more segments 1524 along flexible body 1516, as will be described in further detail below. The tracking system 1530 may include one or more sensors and/or imaging devices. The flexible body 1516, such as the length between the distal end 1518 and the proximal end 1517, may include multiple segments 1524. The tracking system 1530 may be implemented using hardware, firmware, software, or a combination thereof. In some examples, the tracking system 1530 is part of control system 1412.

Tracking system 1530 may track the distal end 1518 and/or one or more of the segments 1524 of the flexible body 1516 using a shape sensor 1522. The shape sensor 1522 may include an optical fiber aligned with the flexible body 1516 (e.g., provided within an interior channel of the flexibly body 1516 or mounted externally along the flexible body 1516). In some examples, the optical fiber may have a diameter of approximately 200 μm. In other examples, the diameter may be larger or smaller. The optical fiber of the shape sensor 1522 may form a fiber optic bend sensor for determining the shape of flexible body 1516. Optical fibers including Fiber Bragg Gratings (FBGs) may be used to provide strain measurements in structures in one or more dimensions. Various systems and methods for monitoring the shape and relative position of an optical fiber in three dimensions, which may be applicable in some embodiments, are described in U.S. Patent Application Publication No. 2006/0013523 (filed Jul. 13, 2005 and titled “Fiber optic position and shape sensing device and method relating thereto”); U.S. Pat. No. 7,772,541 (filed on Mar. 12, 2008 and titled “Fiber Optic Position and/or Shape Sensing Based on Rayleigh Scatter”); and U.S. Pat. No. 8,773,650 (filed on Sep. 2, 2010 and titled “Optical Position and/or Shape Sensing”), which are all incorporated by reference herein in their entireties. Sensors in some embodiments may employ other suitable strain sensing techniques, such as Rayleigh scattering, Raman scattering, Brillouin scattering, and Fluorescence scattering.

In some examples, the shape of the flexible body 1516 may be determined using other techniques. For example, a history of the position and/or pose of the distal end 1518 of the flexible body 1516 can be used to reconstruct the shape of flexible body 1516 over an interval of time (e.g., as the flexible body 1516 is advanced or retracted within a patient anatomy). In some examples, the tracking system 1530 may alternatively and/or additionally track the distal end 1518 of the flexible body 1516 using a position sensor system 1520. Position sensor system 1520 may be a component of an EM sensor system with the position sensor system 1520 including one or more position sensors. Although the position sensor system 1520 is shown as being near the distal end 1518 of the flexible body 1516 to track the distal end 1518, the number and location of the position sensors of the position sensor system 1520 may vary to track different regions along the flexible body 1516. In one example, the position sensors include conductive coils that may be subjected to an externally generated electromagnetic field. Each coil of position sensor system 1520 may produce an induced electrical signal having characteristics that depend on the position and orientation of the coil relative to the externally generated electromagnetic field. The position sensor system 1520 may measure one or more position coordinates and/or one or more orientation angles associated with one or more portions of flexible body 1516. In some examples, the position sensor system 1520 may be configured and positioned to measure six degrees of freedom, e.g., three position coordinates X, Y, Z and three orientation angles indicating pitch, yaw, and roll of a base point. In some examples, the position sensor system 1520 may be configured and positioned to measure five degrees of freedom, e.g., three position coordinates X, Y, Z and two orientation angles indicating pitch and yaw of a base point. Further description of a position sensor system, which may be applicable in some embodiments, is provided in U.S. Pat. No. 6,380,732 (filed Aug. 11, 1999 and titled “Six-Degree of Freedom Tracking System Having a Passive Transponder on the Object Being Tracked”), which is incorporated by reference herein in its entirety.

In some embodiments, the tracking system 1530 may alternately and/or additionally rely on a collection of pose, position, and/or orientation data stored for a point of an elongated device 1502 and/or medical tool 1526 captured during one or more cycles of alternating motion, such as breathing. This stored data may be used to develop shape information about the flexible body 1516. In some examples, a series of position sensors (not shown), such as EM sensors like the sensors in position sensor 1520 or some other type of position sensors may be positioned along the flexible body 1516 and used for shape sensing. In some examples, a history of data from one or more of these position sensors taken during a procedure may be used to represent the shape of elongated device 1502, particularly if an anatomic passageway is generally static.

FIG. 23B is a simplified diagram of the medical tool 1526 within the elongated device 1502 according to some embodiments. The flexible body 1516 of the elongated device 1502 may include the channel 1521 sized and shaped to receive the medical tool 1526. In some embodiments, the medical tool 1526 may be used for procedures such as diagnostics, imaging, surgery, biopsy, ablation, illumination, irrigation, suction, electroporation, etc. Medical tool 1526 can be deployed through channel 1521 of flexible body 1516 and operated at a procedural site within the anatomy. Medical tool 1526 may be, for example, an image capture probe, a biopsy tool (e.g., a needle, grasper, brush, etc.), an ablation tool (e.g., a laser ablation tool, radio frequency (RF) ablation tool, cryoablation tool, thermal ablation tool, heated liquid ablation tool, etc.), an electroporation tool, and/or another surgical, diagnostic, or therapeutic tool. In some examples, the medical tool 1526 may include an end effector having a single working member such as a scalpel, a blunt blade, an optical fiber, an electrode, and/or the like. Other end types of end effectors may include, for example, forceps, graspers, scissors, staplers, clip appliers, and/or the like. Other end effectors may further include electrically activated end effectors such as electrosurgical electrodes, transducers, sensors, and/or the like.

The medical tool 1526 may be a biopsy tool used to remove sample tissue or a sampling of cells from a target anatomic location. In some examples, the biopsy tool is a flexible needle. The biopsy tool may further include a sheath that can surround the flexible needle to protect the needle and interior surface of the channel 1521 when the biopsy tool is within the channel 1521. The medical tool 1526 may be an image capture probe that includes a distal portion with a stereoscopic or monoscopic camera that may be placed at or near the distal end 1518 of flexible body 1516 for capturing images (e.g., still or video images). The captured images may be processed by the visualization system 1531 for display and/or provided to the tracking system 1530 to support tracking of the distal end 1518 of the flexible body 1516 and/or one or more of the segments 1524 of the flexible body 1516. The image capture probe may include a cable for transmitting the captured image data that is coupled to an imaging device at the distal portion of the image capture probe. In some examples, the image capture probe may include a fiber-optic bundle, such as a fiberscope, that couples to a more proximal imaging device of the visualization system 1531. The image capture probe may be single-spectral or multi-spectral, for example, capturing image data in one or more of the visible, near-infrared, infrared, and/or ultraviolet spectrums. The image capture probe may also include one or more light emitters that provide illumination to facilitate image capture. In some examples, the image capture probe may use ultrasound, x-ray, fluoroscopy, CT, MRI, or other types of imaging technology.

In some examples, the image capture probe is inserted within the flexible body 1516 of the elongated device 1502 to facilitate visual navigation of the elongated device 1502 to a procedural site and then is replaced within the flexible body 1516 with another type of medical tool 1526 that performs the procedure. In some examples, the image capture probe may be within the flexible body 1516 of the elongated device 1502 along with another type of medical tool 1526 to facilitate simultaneous image capture and tissue intervention, such as within the same channel 1521 or in separate channels. A medical tool 1526 may be advanced from the opening of the channel 1521 to perform the procedure (or some other functionality) and then retracted back into the channel 1521 when the procedure is complete. The medical tool 1526 may be removed from the proximal end 1517 of the flexible body 1516 or from another optional instrument port (not shown) along flexible body 1516.

In some examples, the elongated device 1502 may include integrated imaging capability rather than utilize a removable image capture probe. For example, the imaging device (or fiber-optic bundle) and the light emitters may be located at the distal end 1518 of the elongated device 1502. The flexible body 1515 may include one or more dedicated channels that carry the cable(s) and/or optical fiber(s) between the distal end 1518 and the visualization system 1531. Here, the medical instrument system 1500 can perform simultaneous imaging and tool operations.

In some examples, the medical tool 1526 is capable of controllable articulation. The medical tool 1526 may house control members or cables (which may also be referred to as pull wires), linkages, or other actuation controls (not shown) that extend between its proximal and distal ends to controllably bend the distal end of medical tool 1526, such as discussed herein for the flexible elongated device 1502. The medical tool 1526 may be coupled to a drive unit 1504 and the manipulator assembly 1402. In these examples, the elongated device 1502 may be excluded from the medical instrument system 1500 or may be a flexible device that does not have controllable articulation. Steerable instruments or tools, applicable in some embodiments, are further described in detail in U.S. Pat. No. 7,316,681 (filed on Oct. 4, 2005 and titled “Articulated Surgical Instrument for Performing Minimally Invasive Surgery with Enhanced Dexterity and Sensitivity”) and U.S. Pat. No. 9,259,274 (filed Sep. 30, 2008 and titled “Passive Preload and Capstan Drive for Surgical Instruments”), which are incorporated by reference herein in their entireties.

The flexible body 1516 of the elongated device 1502 may also or alternatively house cables, linkages, or other steering controls (not shown) that extend between the drive unit 1504 and the distal end 1518 to controllably bend the distal end 1518 as shown, for example, by broken dashed line depictions 1519 of the distal end 1518 in FIG. 23A. In some examples, at least four cables are used to provide independent up-down steering to control a pitch of the distal end 1518 and left-right steering to control a yaw of the distal end 1518. In these examples, the flexible elongated device 1502 may be a steerable catheter. Examples of steerable catheters, applicable in some embodiments, are described in detail in PCT Publication WO 2019/018736 (published J an. 24, 2019 and titled “Flexible Elongated Device Systems and Methods”), which is incorporated by reference herein in its entirety.

In embodiments where the device 1502 and/or medical tool 1526 are actuated by a robot-assisted assembly (e.g., the manipulator assembly 1402), the drive unit 1504 may include drive inputs that removably couple to and receive power from drive elements, such as actuators, of the robot-assisted assembly. In some examples, the elongated device 1502 and/or medical tool 1526 may include gripping features, manual actuators, or other components for manually controlling the motion of the elongated device 1502 and/or medical tool 1526. The elongated device 1502 may be steerable or, alternatively, the elongated device 1502 may be non-steerable with no integrated mechanism for operator control of the bending of distal end 1518. In some examples, one or more channels 1521 (which may also be referred to as lumens), through which medical tools 1526 can be deployed and used at a target anatomical location, may be defined by the interior walls of the flexible body 1516 of the elongated device 1502.

In some examples, the medical instrument system 1500 (e.g., the elongated device 1502 or medical tool 1526) may include a flexible bronchial instrument, such as a bronchoscope or bronchial catheter, for use in examination, diagnosis, biopsy, and/or treatment of a lung. The medical instrument system 1500 may also be suited for navigation and treatment of other tissues, via natural or surgically created connected passageways, in any of a variety of anatomic systems, including the colon, the intestines, the kidneys and kidney calices, the brain, the heart, the circulatory system including vasculature, and/or the like.

The information from the tracking system 1530 may be sent to the navigation system 1532, where the information may be combined with information from the visualization system 1531 and/or pre-operatively obtained models to provide the physician, clinician, surgeon, or other operator with real-time position information. In some examples, the real-time position information may be displayed on the display system 1410 for use in the control of the medical instrument system 1500. In some examples, the navigation system 1532 may utilize the position information as feedback for positioning medical instrument system 1500. Various systems for using fiber optic sensors to register and display a surgical instrument with surgical images, applicable in some embodiments, are provided in U.S. Pat. No. 8,900,131 (filed May 13, 2011 and titled “Medical System Providing Dynamic Registration of a Model of an Anatomic Structure for Image-Guided Surgery”), which is incorporated by reference herein in its entirety.

In the description, specific details have been set forth describing some examples. Numerous specific details are set forth in order to provide a thorough understanding of the examples. It will be apparent, however, to one skilled in the art that some examples may be practiced without some or all of these specific details. The specific examples disclosed herein are meant to be illustrative but not limiting. One skilled in the art may realize other elements that, although not specifically described here, are within the scope and the spirit of this disclosure.

Elements described in detail with reference to one example, implementation, or application optionally may be included, whenever practical, in other examples, implementations, or applications in which they are not specifically shown or described. For example, if an element is described in detail with reference to one example and is not described with reference to a second example, the element may nevertheless be claimed as included in the second example. Thus, to avoid unnecessary repetition in the following description, one or more elements shown and described in association with one example, implementation, or application may be incorporated into other examples, implementations, or aspects unless specifically described otherwise, unless the one or more elements would make an example or implementation non-functional, or unless two or more of the elements provide conflicting functions.

Any alterations and further modifications to the described devices, instruments, methods, and any further application of the principles of the present disclosure are fully contemplated as would normally occur to one skilled in the art to which the disclosure relates. In particular, it is fully contemplated that the features, components, and/or steps described with respect to one example may be combined with the features, components, and/or steps described with respect to other examples of the present disclosure. In addition, dimensions provided herein are for specific examples and it is contemplated that different sizes, dimensions, and/or ratios may be utilized to implement the concepts of the present disclosure. To avoid needless descriptive repetition, one or more components or actions described in accordance with one illustrative example can be used or omitted as applicable from other illustrative examples. For the sake of brevity, the numerous iterations of these combinations will not be described separately. For simplicity, in some instances the same reference numbers are used throughout the drawings to refer to the same or like parts.

The systems and methods described herein may be suited for imaging, via natural or surgically created connected passageways, in any of a variety of anatomic systems, including the lung, colon, the intestines, the stomach, the liver, the kidneys and kidney calices, the brain, the heart, the circulatory system including vasculature, and/or the like. While some examples are provided herein with respect to medical procedures, any reference to medical or surgical instruments and medical or surgical methods is non-limiting. For example, the instruments, systems, and methods described herein may be used for non-medical purposes including industrial uses, general robotic uses, and sensing or manipulating non-tissue work pieces. Other example applications involve cosmetic improvements, imaging of human or animal anatomy, gathering data from human or animal anatomy, and training medical or non-medical personnel. Additional example applications include use for procedures on tissue removed from human or animal anatomies (without return to a human or animal anatomy) and performing procedures on human or animal cadavers. Further, these techniques can also be used for surgical and nonsurgical medical treatment or diagnosis procedures.

The methods described herein are illustrated as a set of operations or processes. Not all the illustrated processes may be performed in all examples of the methods. Additionally, one or more processes that are not expressly illustrated or described may be included before, after, in between, or as part of the example processes. In some examples, one or more of the processes may be performed by the control system (e.g., control system 1412) or may be implemented, at least in part, in the form of executable code stored on non-transitory, tangible, machine-readable media that when run by one or more processors (e.g., the processors 1414 of control system 1412) may cause the one or more processors to perform one or more of the processes.

One or more components of the embodiments discussed in this disclosure, such as control system 1412, may be implemented in software for execution on one or more processors of a computer system. The software may include code that when executed by the one or more processors, configures the one or more processors to perform various functionalities as discussed herein. The code may be stored in a non-transitory computer readable storage medium (e.g., a memory, magnetic storage, optical storage, solid-state storage, etc.). The computer readable storage medium may be part of a computer readable storage device, such as an electronic circuit, a semiconductor device, a semiconductor memory device, a read only memory (ROM), a flash memory, an erasable programmable read only memory (EPROM); a floppy diskette, a CD-ROM, an optical disk, a hard disk, or other storage device. The code may be downloaded via computer networks such as the Internet, Intranet, etc. for storage on the computer readable storage medium. The code may be executed by any of a wide variety of centralized or distributed data processing architectures. The programmed instructions of the code may be implemented as a number of separate programs or subroutines, or they may be integrated into a number of other aspects of the systems described herein. The components of the computing systems discussed herein may be connected using wired and/or wireless connections. In some examples, the wireless connections may use wireless communication protocols such as Bluetooth, near-field communication (NFC), Infrared Data Association (IrDA), home radio frequency (HomeRF), IEEE 802.11, Digital Enhanced Cordless Telecommunications (DECT), and wireless medical telemetry service (WMTS).

Note that the processes and displays presented may not inherently be related to any particular computer or other apparatus. Various general-purpose systems may be used with programs in accordance with the teachings herein, or it may prove convenient to construct a more specialized apparatus to perform the operations described. The required structure for a variety of these systems will appear as elements in the claims. In addition, the examples of the invention are not described with reference to any particular programming language. It will be appreciated that a variety of programming languages may be used to implement the teachings of the invention as described herein.

In some instances well known methods, procedures, components, and circuits have not been described in detail so as not to unnecessarily obscure aspects of the examples. This disclosure describes various instruments, portions of instruments, and anatomic structures in terms of their state in three-dimensional space. As used herein, the term “position” refers to the location of an object or a portion of an object in a three-dimensional space (e.g., three degrees of translational freedom along Cartesian x-, y-, and z-coordinates). As used herein, the term “orientation” refers to the rotational placement of an object or a portion of an object (three degrees of rotational freedom—e.g., roll, pitch, and yaw). As used herein, the term “pose” refers to the position of an object or a portion of an object in at least one degree of translational freedom and to the orientation of that object or portion of the object in at least one degree of rotational freedom (up to six total degrees of freedom). As used herein, the term “shape” refers to a set of poses, positions, or orientations measured along an object. As used herein, the term “distal” refers to a position that is closer to a procedural site and the term “proximal” refers to a position that is further from the procedural site. Accordingly, the distal portion or distal end of an instrument is closer to a procedural site than a proximal portion or proximal end of the instrument when the instrument is being used as designed to perform a procedure.

While certain exemplary examples of the invention have been described and shown in the accompanying drawings, it is to be understood that such examples are merely illustrative of and not restrictive on the broad invention, and that the examples of the invention not be limited to the specific constructions and arrangements shown and described, since various other alternatives, modifications, and equivalents will be appreciated by those with ordinary skill in the art.

1. A system comprising:
a flexible elongated device including:
a fluid channel and a pore extending between the fluid channel and a surface of the flexible elongated device; and
a lubricious layer extending over at least a portion of the surface of the flexible elongated device;
a fluid system coupled to the flexible elongated device; and
a control system configured to: cause a fluid to be released by the fluid system to the fluid channel of the flexible elongated device, the fluid in the fluid channel flowing through the pore to transition the lubricious layer from a dehydrated condition to a hydrated condition.

2. The system of claim 1, wherein the control system is configured to:
determine the dehydrated condition of the flexible elongated device; and
cause the fluid to be released by the fluid system in response to the dehydrated condition.

3. The system of claim 1, wherein the pore extends through the lubricious layer.

4. The system of claim 1, wherein the pore extends to the lubricious layer.

5. The system of claim 1, wherein a hydration reservoir is disposed between the fluid channel and the pore.

6. The system of claim 5, wherein the hydration reservoir is within an open cell foam.

7. The system of claim 1, wherein the pore has an elongated opening.

8. The system of claim 1, wherein the pore has a round opening.

9. The system of claim 1, wherein the pore is one in a series of pores extending between the fluid channel and the surface of the flexible elongated device.

10. The system of claim 9, wherein the series of pores are arranged linearly along a length of the flexible elongated device.

11. The system of claim 9, wherein the series of pores are arranged radially around a diameter of the flexible elongated device.

12. The system of claim 9, wherein the series of pores are arranged spirally around the flexible elongated device.

13. The system of claim 1, wherein the pore extends at a non-orthogonal angle to a longitudinal axis of the flexible elongated device.

14. The system of claim 1, wherein a flexible elastomeric layer of the flexible elongated device includes the surface of the flexible elongated device and wherein the flexible elastomeric layer includes an active polymer component that expands to cause migration of a fluid in the pore to the surface of the flexible elongated device in response to a stimulus.

15. The system of claim 14, wherein the stimulus is a ferroelectric stimulus.

16. The system of claim 14, wherein the stimulus is a piezoelectric stimulus.

17. The system of claim 14, wherein the control system is further configured to cause fluid to be released through the pore in response to a detected dehydrated threshold level by stimulating the active polymer component.

18. A system comprising:
a flexible elongated device configured to couple to a robotically-assisted manipulator assembly, the flexible elongated device including a lubricious layer extending over at least a portion of a surface of the flexible elongated device;
a hydration coupling system connected to the robotically-assisted manipulator assembly; and
a control system configured to cause a fluid to be released through the hydration coupling system into contact with an external surface of the flexible elongated device to transition the lubricious layer from a dehydrated condition to a hydrated condition.

19. The system of claim 18, wherein the control system is configured to:
determine the dehydrated condition of the flexible elongated device and
cause the fluid to be released in response to the dehydrated condition.

20. The system of claim 18, further comprising a fluid system coupled to the hydration coupling system.

21-57. (canceled)
